(19) United States
(12) Patent Application Publication
(10) Pub. No.: US 2019/0239860 A1
(43) Pub. Date: Aug. 8, 2019

(54) APPARATUS, METHOD AND PROGRAM FOR DISPLAYING ULTRASOUND IMAGE AND PHOTOACOUSTIC IMAGE

(71) Applicant: CANON KABUSHIKI KAISHA, Tokyo (JP)

(72) Inventors: Akinori Hayashi, Tokyo (JP); Nobu Miyazawa, Yokohama-shi (JP); Shota Yamada, Kawasaki-shi (JP); Daiya Semba, Inagi-shi (JP); Yusuke Nojiri, Tokyo (JP); Kazuhito Oka, Tokyo (JP)

(21) Appl. No.: 16/315,542

(22) PCT Filed: Jun. 26, 2017
(86) PCT No.: PCT/JP2017/023344

§ 371 Date: Jan. 4, 2019

(30) Foreign Application Priority Data
Jul. 8, 2016 (JP) 2016-136104

(51) Int. Cl. A61B 8/08; A61B 5/00; A61B 8/00

(52) U.S. Cl. CPC A61B 8/5261; A61B 8/54; A61B 8/463; A61B 5/0095

(57) ABSTRACT

An apparatus according to the present invention includes a display control unit configured to control to display an ultrasound image on a first display area in a display unit. In this case, the display control unit controls to, based on information representing a display instruction given when a moving image of the ultrasound image is being displayed, display a still image of a superimposition image of the ultrasound image and a photoacoustic image corresponding to a time point of the display instruction on a second display area different from the first display area in the display unit and, on the other hand, continue the display of the moving image of the ultrasound image on the first display area.

### TECHNICAL FIELD

The present invention relates to an apparatus or a method for displaying an ultrasound image and a photoacoustic image.

### BACKGROUND ART

An ultrasonic diagnostic apparatus which generates an ultrasound image by transmitting and receiving ultrasonic waves has been known as an image diagnostic apparatus which images an internal state of a living body noninvasively. An ultrasonic diagnostic apparatus generates an ultrasound image on the basis of a reception signal of transmitted waves or reflected waves (ultrasonic echo) of transmission ultrasonic waves.

On the other hand, a photoacoustic apparatus which applies an ultrasonic wave (photoacoustic wave) generated by biological tissue irradiated with light and adiabatically expanded due to optical energy of the irradiated light has been known as an image diagnostic apparatus which images an internal state of a living body noninvasively. Such a photoacoustic apparatus may generate a photoacoustic image based on a reception signal of photoacoustic waves.

PTL 1 discloses a switch for selecting an operation mode in which a reflected ultrasonic wave is detected or an operation mode in which photoacoustic waves are detected. PTL 1 discloses selecting display of an ultrasound image or display of a photoacoustic image by using the switch.

### CITATION LIST

### Patent Literature

PTL 1: Japanese Patent Laid-Open No. 2012-196430

### SUMMARY OF INVENTION

### Technical Problem

For diagnoses using ultrasound images and photoacoustic images, it is assumed that there is a need for using an ultrasound image as a fundamental diagnosis image and displaying a photoacoustic image for aiding diagnosis based on the ultrasound image, like an ultrasonic diagnostic apparatus in the past. In this case, when display of an ultrasonic image is changed to display of a photoacoustic image or a superimposition image, as disclosed in PTL 1, the fundamental diagnosis based on the ultrasound image may possibly be hindered.

The present invention provides an apparatus including a first obtaining unit configured to obtain an ultrasound image generated by transmitting and receiving ultrasonic waves to and from an object, a second obtaining unit configured to obtain a photoacoustic image generated based on photoacoustic waves generated from light irradiated to the object, and a display control unit configured to control to display the ultrasound image on a first display area in a display unit, wherein the display control unit controls to, based on information representing a display instruction given when a moving image of the ultrasound image is being displayed, display a still image of a superimposition image of the ultrasound image and the photoacoustic image corresponding to a time point of the display instruction on a second display area different from the first display area in the display unit and, on the other hand, continue the display of the moving image of the ultrasound image on the first display area.

Further features of the present invention will become apparent from the following description of exemplary embodiments with reference to the attached drawings.

### BRIEF DESCRIPTION OF DRAWINGS

FIG. 1 is a block diagram illustrating an inspection system according to a first embodiment.

FIG. 2 is a schematic diagram illustrating a probe according to the first embodiment.

FIG. 3 is a configuration diagram illustrating a computer and peripherals therefor according to the first embodiment.

FIG. 4 is a flowchart illustrating a saving method according to the first embodiment.

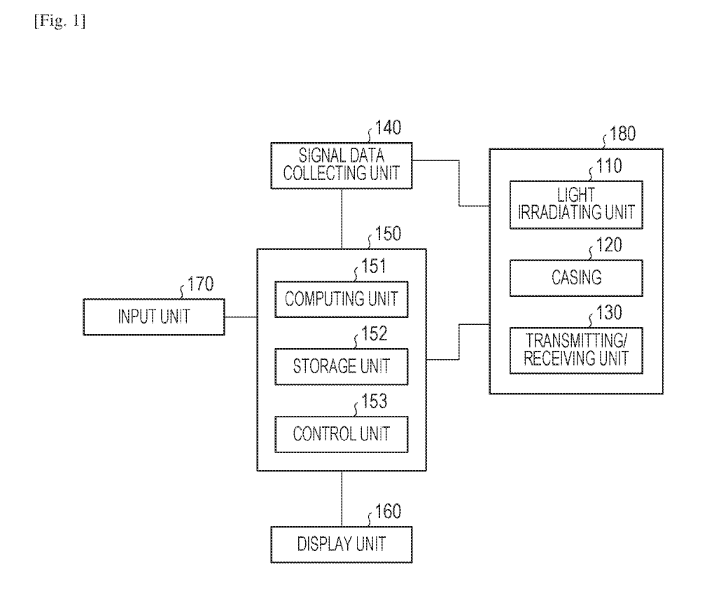

FIG. 1 illustrates a data structure of saved data according to the first embodiment.

FIG. 6 is a timing chart according to the first embodiment.

FIG. 7 is another timing chart according to the first embodiment.

FIG. 8 is another timing chart according to the first embodiment.

FIG. 9 is another timing chart according to the first embodiment.

FIG. 10 is a flowchart illustrating a saving method according to a second embodiment.

FIG. 11 illustrates a timing chart according to the second embodiment.

FIG. 12 is another timing chart according to the second embodiment.

FIG. 13 is another timing chart according to the second embodiment.

FIG. 14 is another timing chart according to the second embodiment.

FIG. 15 illustrates a data structure of examination order information according to a third embodiment.

FIG. 16A illustrates a display example of a display unit according to a fourth embodiment.

FIG. 16B illustrates the display example of a display unit according to the fourth embodiment.

FIG. 16C illustrates the display example of a display unit according to the fourth embodiment.

FIG. 17A illustrates another display example of a display unit according to the fourth embodiment.

FIG. 17B illustrates the display example of a display unit according to the fourth embodiment.

FIG. 17C illustrates the display example of a display unit according to the fourth embodiment.

FIG. 18A illustrates another display example of a display unit according to the fourth embodiment.

FIG. 18B illustrates the display example of a display unit according to the fourth embodiment.

FIG. 19A illustrates another display example of a display unit according to the fourth embodiment.

FIG. 19B illustrates the display example of a display unit according to the fourth embodiment.

FIG. 20A illustrates a display image example according to the fourth embodiment.

FIG. 20B illustrates another display image example according to the fourth embodiment.

FIG. 20C illustrates another display image example according to the fourth embodiment.

FIG. 21 illustrates an example of a GUI according to the fourth embodiment.

FIG. 22 illustrates another example of the GUI according to the fourth embodiment.

### DESCRIPTION OF EMBODIMENTS

For convenience of description, an acoustic wave generated by thermal expansion of an optical absorber irradiated with light will be called a photoacoustic wave, hereinafter. Furthermore, for convenience of description, an acoustic wave or a reflected wave (echo) transmitted from a transducer will be called an ultrasonic wave, hereinafter.

### First Embodiment

Embodiments of the present invention will be described in detail below with reference to drawings. Like numbers refer to like parts throughout in principle, and any repetitive description will be omitted.

Use of a superimposition image of an ultrasound image and a photoacoustic image is considered as effective for diagnoses. Accordingly, it may also be considered as effective for diagnoses that obtaining and saving an ultrasound image and a photoacoustic image with a smaller time difference therebetween and displaying the images associated with each other in a superimposed or parallel arrangement.

On the other hand, like an ultrasonic diagnostic apparatus in the past, a user such as a doctor or a technician may prefer to instruct to save an ultrasound image by checking a display image thereof. In this case, if a photoacoustic image is superimposed on an ultrasound image, there is a possibility that the photoacoustic image may prevent a user from determining whether to instruct to save or not.

A saving method for an ultrasonic diagnostic apparatus in the past may require saving an ultrasound image first, then changing the display image from the ultrasound image to a photoacoustic image, and saving the photoacoustic image.

Accordingly, when an instruction to save (hereinafter, save instruction) is given when a moving image of an ultrasound image is displayed when a photoacoustic image is not displayed, this embodiment saves the photoacoustic image corresponding to the save instruction in addition to the ultrasound image corresponding to the save instruction. That is, the photoacoustic image and the ultrasound image are stored in a storage unit in response to the save instruction. For example, an ultrasound image displayed when a save instruction is given and a photoacoustic image neighboring in time to the time point when the ultrasound image is obtained are saved in association. Then, a still image of a superimposition image of the associated ultrasound image and photoacoustic image is displayed. Thus, when a user needs to save a photoacoustic image when checking an ultrasound image, the user can save the photoacoustic image and the ultrasound image in association without requiring changing the display image to the photoacoustic image. Therefore, the user can check a superimposition image of the photoacoustic image and the ultrasound image obtained with a small time difference therebetween even after an inspection. Furthermore, according to this embodiment, when an instruction is given from a user, a still image of the superimposition image is displayed, and, on the other hand, the display of the moving image of the ultrasound image is continued. This can suppress hindrance against basic diagnosis based on the ultrasound image and allows display of the still image of the photoacoustic image.

### Configuration of Inspection System

An inspection system according to a first embodiment will be schematically described with reference to FIG. 1. FIG. 1 is a schematic block diagram illustrating an overall inspection system. The inspection system according to this embodiment includes a signal data collecting unit 140, a computer 150, a display unit 160, an input unit 170, and a probe 180.

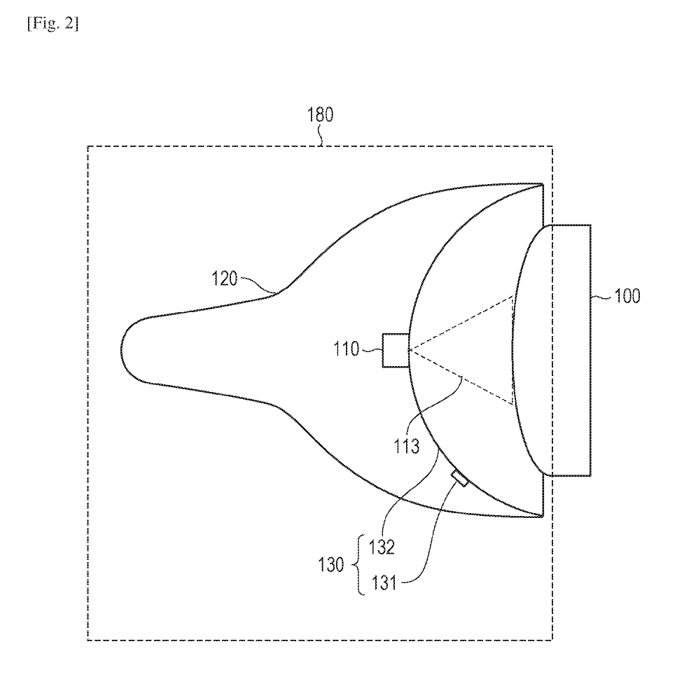

FIG. 2 is a schematic diagram of the probe 180 according to this embodiment. The probe 180 has a light irradiating unit 110, a casing 120 including a holding portion, and a transmitting/receiving unit 130. An object 100 is a measurement object.

The light irradiating unit 110 irradiates pulsed light 113 to the object 100 so that acoustic waves can occur within the object 100. An acoustic wave caused by light due to a photoacoustic effect will also be called a photoacoustic wave. The transmitting/receiving unit 130 is configured to receive photoacoustic waves and output an analog electric signal (photoacoustic signal). The transmitting/receiving unit 130 is further configured to transmit ultrasonic waves to the object 100 and receive echo waves of the transmitted ultrasonic waves to output an analog electric signal (ultrasonic signal).

The signal data collecting unit 140 is configured to convert an analog signal output from the transmitting/receiving unit 130 to a digital signal and output it to the computer 150. The computer 150 stores the digital signal output from the signal data collecting unit 140 as signal data derived from ultrasonic waves or photoacoustic waves.

The computer 150 is configured to perform signal processing on a stored digital signal to generate image data representing an ultrasound image or a photoacoustic image. The computer 150 performs an image process on the resulting image data and then outputs image data to the display unit 160. The display unit 160 is configured to display an ultrasound image or a photoacoustic image. A doctor or a technician as a user can perform diagnosis by checking an ultrasound image and a photoacoustic image displayed on the display unit 160. A display image is saved in a data management system connected to a memory within the computer 150 or to the inspection system over a network based on a save instruction from a user or the computer 150.

The computer 150 is configured to perform drive control over components included in the inspection system. The display unit 160 may display an image generated in the computer 150 and a GUI. The input unit 170 is configured to be usable by a user for inputting information. A user may use the input unit 170 to perform an operation such as instructing to save a display image.

A photoacoustic image obtained by the inspection system according to this embodiment is a concept including an image derived from photoacoustic waves generated from irradiated light. A photoacoustic image includes image data representing at least one spatial distribution of information regarding sound pressure for generating photoacoustic waves (initial sound pressure), an optical absorption energy density, an optical absorption coefficient, and a concentration of a substance contained in an object, for example. The information regarding a concentration of a substance may be an oxyhemoglobin concentration, a deoxyhemoglobin concentration, a total hemoglobin concentration, or an oxygen saturation, for example. The total hemoglobin concentration is a sum of an oxyhemoglobin concentration and a deoxyhemoglobin concentration. The oxygen saturation is a ratio of oxyhemoglobin to whole hemoglobin. The photoacoustic image is not limited to an image representing a spatial distribution but may be an image representing a numerical value. For example, the photoacoustic image is a concept including an image representing information derived from a photoacoustic signal, such as a photoacoustic signal (RAW data), an average concentration of a substance contained in an object, a pixel value at a specific position in a spatial distribution, or a statistic (such as an average value or a median value) of pixel values in a spatial distribution, for example. As a photoacoustic image, a numerical value of an average concentration of a substance contained in an object, for example, may be displayed on the display unit 160.

An ultrasound image obtained by the inspection system according to this embodiment includes image data of at least one of a B mode image, a doppler image, and an elastography image. The ultrasound image is a concept including an image obtained by transmitting and receiving ultrasonic waves.

Components of an object information obtaining apparatus according to this embodiment will be described in detail below.

### Light Irradiating Unit 110

The light irradiating unit 110 includes a light source configured to emit pulsed light 113, and an optical system configured to guide the pulsed light 113 emitted from the light source to the object 100. The pulsed light here includes so-called square-wave or triangle-wave light.

The light emitted from the light source may have a pulse width ranging from 1 ns to 100 ns. The light may have a wavelength ranging from 400 nm to 1600 nm. In order to image a blood vessel neighboring to a surface of a living body with a high resolution, light having a wavelength (ranging from 400 nm to 700 nm) which is largely absorbed by a blood vessel may be used. On the other hand, in order to image a deep part of a living body, light having a wavelength (ranging from 700 nm to 1100 nm) which is typically absorbed less by background tissue (such as water or fat) of a living body may be used.

The light source may be a laser or a light emitting diode, for example. Alternatively, the light source may be capable of performing wavelength conversion for measurement using light having a plurality of wavelengths. When light having a plurality of wavelengths is irradiated to an object, a plurality of light sources which emit light beams having wavelengths different from each other may be provided so that the light beams can be irradiated alternately from the light sources. A set of a plurality of light sources if used is also collectively called as a light source. Various lasers may be applied here such as a solid-state laser, a gas laser, a dye laser, and a semiconductor laser. For example, a pulsed laser such as an Nd:YAG laser and an alexandrite laser may be used as the light source 111. Alternatively, a Ti:sa laser or an OPO (Optical Parametric Oscillators) laser applying an Nd:YAG laser light as excited light may be used as the light source. A microwave source may be used as the light source instead.

The optical system may include optical elements such as a lens, a mirror, and optical fiber. In a case where a breast is the object 100, for example, pulsed light having an increased beam diameter is to be irradiated. Accordingly, the optical system may include a light emitting unit having a diffusing plate configured to diffuse light. On the other hand, an photoacoustic microscope may have an increased resolution with an optical system having a light emitting unit including a lens to irradiate a focused beam.

Alternatively, the pulsed light 113 may be irradiated from the light source directly to the object 100 by the light irradiating unit 110 without an optical system. The components of the light irradiating unit 110 such as the light source may be provided externally to the casing 120.

### Transmitting/Receiving Unit 130

The transmitting/receiving unit 130 includes a transducer 131 configured to output an electric signal from received acoustic waves, and a supporting member 132 configured to support the transducer 131. The transducer 131 is also capable of transmitting acoustic waves. FIG. 2 only illustrates one transducer 131 for simplicity, the transmitting/receiving unit 130 may include a plurality of transducers.

The transducer 131 may be formed of a piezoelectric ceramic material such as PZT (lead zirconate titanate) or a polymer piezoelectric film material such as PVDF (polyvinylidene difluoride), for example. An element excluding a piezoelectric element may be used instead. For example, capacitive micro-machined ultrasonic transducers, CMUT, or a transducer applying a Fabry-Perot interferometer may be used. Any kind of transducer may be adopted if it is capable of outputting an electric signal from received acoustic waves. A signal obtained by the transducer is a temporal resolution signal. In other words, a signal obtained by a receiving element has an amplitude representing a value (such as a value proportional to sound pressure) based on sound pressure received by the transducer at different times.

Photoacoustic waves contain frequency components typically ranging from 100 KHz to 100 MHz, and the transducer 131 is capable of detecting these frequencies.

The supporting member 132 may be formed of a metallic material having a high mechanical strength. For a case where a user holds the casing 120 to scan the probe 180, the supporting member 132 may be formed of a polymer material such as plastics from view point of weight reduction. In order to launch more irradiation light into an object, the supporting member 132 may have a mirror surface or a surface processed to be light scattering closer to the object 100. According to this embodiment, the supporting member 132 has a hemispherical enclosure shape and is configured to support a plurality of transducers 131 on the hemispherical enclosure. In this case, the transducers 131 arranged on the supporting member 132 have directional axes gathering closely to the center of the curvature of the hemisphere. An image obtained by using a group of electric signals output from the plurality of transducers 131 has high image quality at a part produced by electric signals from the transducers around the center of curvature. The supporting member 132 may have any configuration if it can support the transducers 131. The supporting member 132 may have a plurality of transducers on its plane or curved surface such as a 1D array, a 1.5D array, a 1.75D array, and a 2D array.

The supporting member 132 may function as a container configured to reserve an acoustic matching material. In other words, the supporting member 132 may be a container for arranging an acoustic matching material between the transducer 131 and the object 100.

The transmitting/receiving unit 130 may include an amplifier configured to amplify time-series analog signals output from the transducers 131. The transmitting/receiving unit 130 may include an A/D converter configured to convert time-series analog signals output from the transducers 131 to time-series digital signals. In other words, the transmitting/receiving unit 130 may include a signal data collecting unit 140, which will be described below.

For detection of acoustic waves at various angles, the transducer 131 may be arranged to surround the entire perimeter of the object 100. However, in a case where it is difficult to arrange transducers to surround the entire perimeter of the object 100, the transducers may be arranged on the hemisphere supporting member to surround the entire perimeter as illustrated in FIG. 2.

The arrangement and number of transducers and the shape of the supporting member may be optimized in accordance with an object, and any kind of transmitting/receiving unit 130 may be adopted with respect to the present invention.

The space between the transmitting/receiving unit 130 and the object 100 is filled with a medium in which photoacoustic waves can propagate. The medium may be made of a material in which acoustic waves can propagate and which has an acoustic characteristic matching at an interface between the object 100 and the transducer 131 and has a transmittance of photoacoustic waves as high as possible. For example, the medium may be water or ultrasound gel.

It should be noted that a transducer configured to transmit ultrasonic waves and a transducer configured to receive acoustic waves may be provided separately. Alternatively, one transducer may be provided which is configured to transmit ultrasonic waves and receive acoustic waves. A transducer configured to transmit and receive ultrasonic waves and a transducer configured to receive photoacoustic waves may be provided separately. Alternatively, one transducer may be provided which is configured to transmit and receive ultrasonic waves and receive photoacoustic waves.

### Signal Data Collecting Unit 140

The signal data collecting unit 140 includes an amplifier configured to amplify an electric signal being an analog signal output from the transducer 131 and an A/D converter configured to convert an analog signal output from the amplifier to a digital signal. The signal data collecting unit 140 may be an FPGA (Field Programmable Gate Array) chip, for example. A digital signal output from the signal data collecting unit 140 is stored in a storage unit 152 within the computer 150. The signal data collecting unit 140 is also called a Data Acquisition System (DAS). The term “electric signal” herein refers to a concept including an analog signal and a digital signal. The signal data collecting unit 140 is connected to a light detection sensor attached to the light emitting unit in the light irradiating unit 110 and may start processing by being triggered by and synchronized with emission of the pulsed light 113 from the light irradiating unit 110. The signal data collecting unit 140 may start the processing by being triggered by and synchronized with a save instruction given by using a freeze button, which will be described below.

### Computer 150

The computer 150 includes a computing unit 151, the storage unit 152, and a control unit 153. These components have functions, which will be described with reference to a processing flow.

A unit responsible for a computing function as the computing unit 151 may have a processor such as a CPU and a GPU (Graphics Processing Unit) and a computing circuit such as an FPGA (Field Programmable Gate Array) chip. These units may include a plurality of processors and computing circuits, instead of a single processor and a single computing circuit. The computing unit 151 may process a reception signal in accordance with parameters such as the speed of sound of an object and a holding cup from the input unit 170.

The storage unit 152 may be a non-transitory storage medium such as a ROM (Read only memory), a magnetic disk and a flash memory. The storage unit 152 may be a volatile medium such as a RAM (Random Access Memory). A storage medium storing a program is a non-transitory storage medium.

The control unit 153 is configured by a computing element such as a CPU. The control unit 153 is configured to control operations performed by components of the photoacoustic apparatus. The control unit 153 may control the components of the inspection system in response to an instruction signal based on an operation such as a start of measurement given through the input unit 170. The control unit 153 may read out program code stored in the storage unit 152 and controls an operation performed by a component of the inspection system.

The computer 150 may be a specially designed workstation. The components of the computer 150 may be configured by different hardware modules. Alternatively, at least partial components of the computer 150 may be configured by a single hardware module.

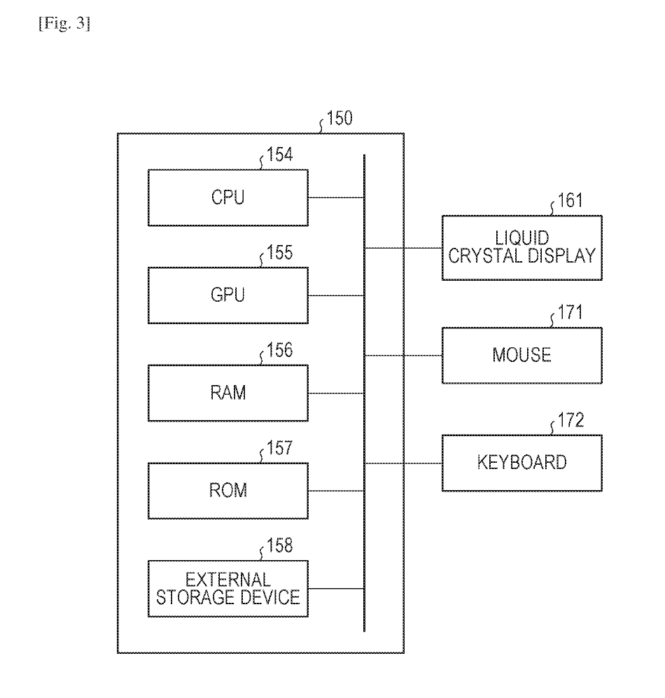

FIG. 3 illustrates a specific configuration example of the computer 150 according to this embodiment. The computer 150 according to this embodiment includes a CPU 154, a GPU 155, a RAM 156, a ROM 157, and an external storage device 158. A liquid crystal display 161 as the display unit 160 and a mouse 171 and a keyboard 172 as the input unit 170 are connected to the computer 150.

The computer 150 and the plurality of transducers 131 may be accommodated in a common casing. Alternatively, partial signal processing may be performed by the computer accommodated in the casing while the rest of the signal processing may be performed by a computer provided externally to the casing. In this case, the computers provided internally and externally to the casing may be collectively called a computer according to this embodiment.

### Display Unit 160

The display unit 160 is a display such as a liquid crystal display and an organic EL (Electro Luminescence). The display unit 160 is configured to display an image based on object information obtained by the computer 150 and a numerical value corresponding to a specific position therein. The display unit 160 may display a graphical user interface (GUI) usable for operating an image or the system. For display of object information, the display unit 160 or the computer 150 may perform an image process (such as adjustment of a luminance value) thereon.

### Input Unit 170

The input unit 170 may be an operating console which can be operated by a user and may include a mouse and a keyboard. The display unit 160 may include a touch panel so that the display unit 160 can also be used as the input unit 170. The input unit 170 may include a freeze button usable by a user for giving an instruction such as a save instruction, which will be described below.

The components of the inspection system may be provided as separate apparatuses or may be integrated to one system. Alternatively, at least partial components of the inspection system may be integrated to one apparatus.

### Object 100

The object 100 will be described below though it is not a component of the inspection system. The inspection system according to this embodiment is usable for purposes such as diagnoses of human or animal malignant tumors and blood vessel diseases and follow-ups of chemical treatments. Therefore, the object 100 is assumed as a region to be diagnosed such as a living body, more specifically, the limbs including the breast, the neck, the abdomen, a finger and a toe of a human body or an animal. For example, in a case where a human body is a measurement object, oxyhemoglobin or deoxyhemoglobin or a blood vessel mostly including them or a neovessel formed in neighborhood of a tumor may be an optical absorber. Plaque of a carotid artery wall may be an optical absorber. Alternatively, a pigment such as methylene blue (MB), indocyanine green (ICG), gold minute particles, or an externally introduced substance integrating or chemically modifying them may be an optical absorber.

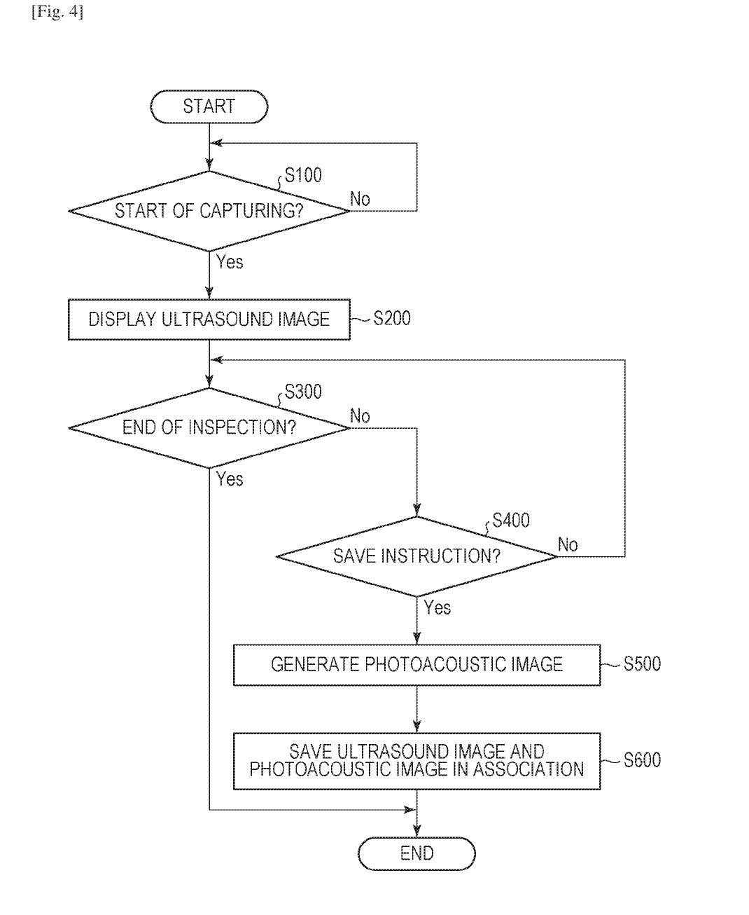

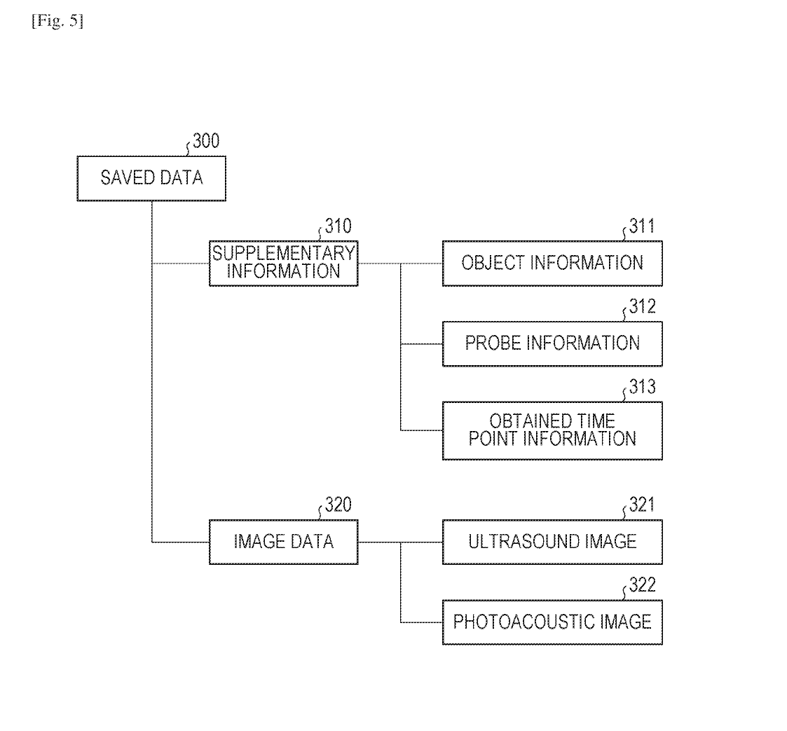

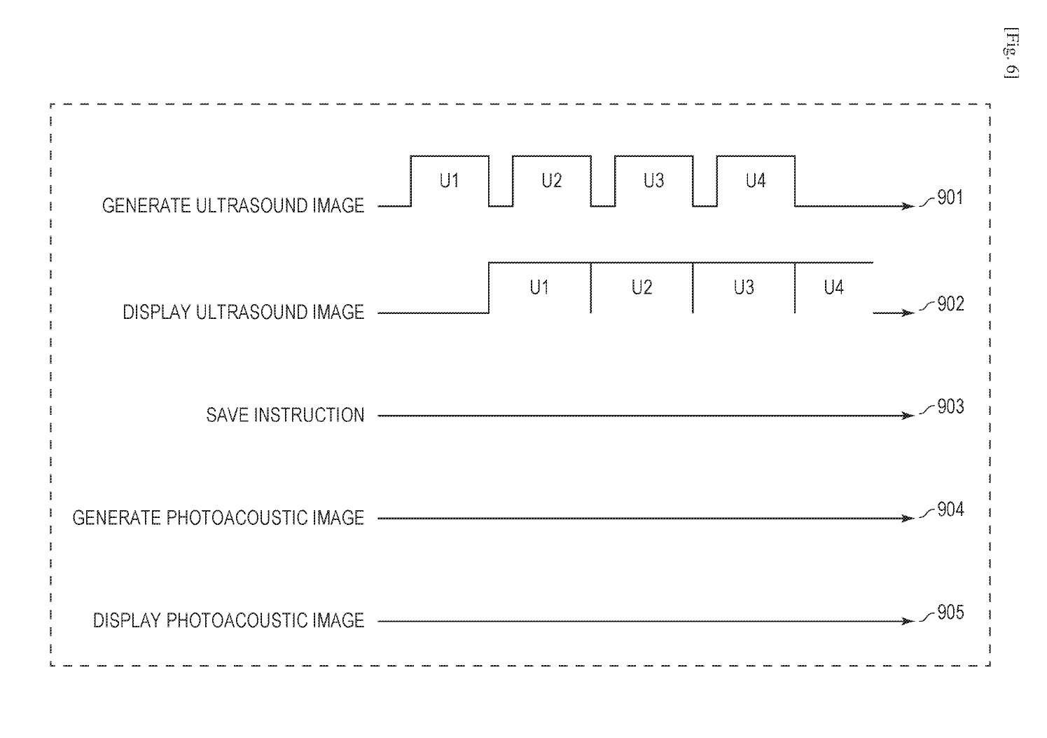

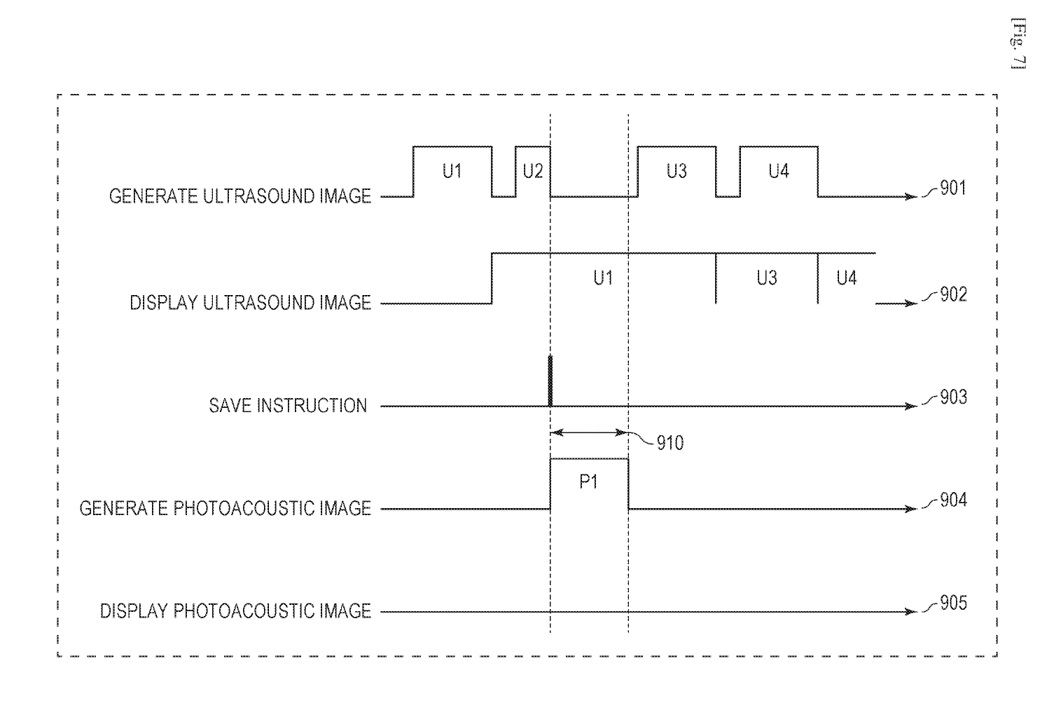

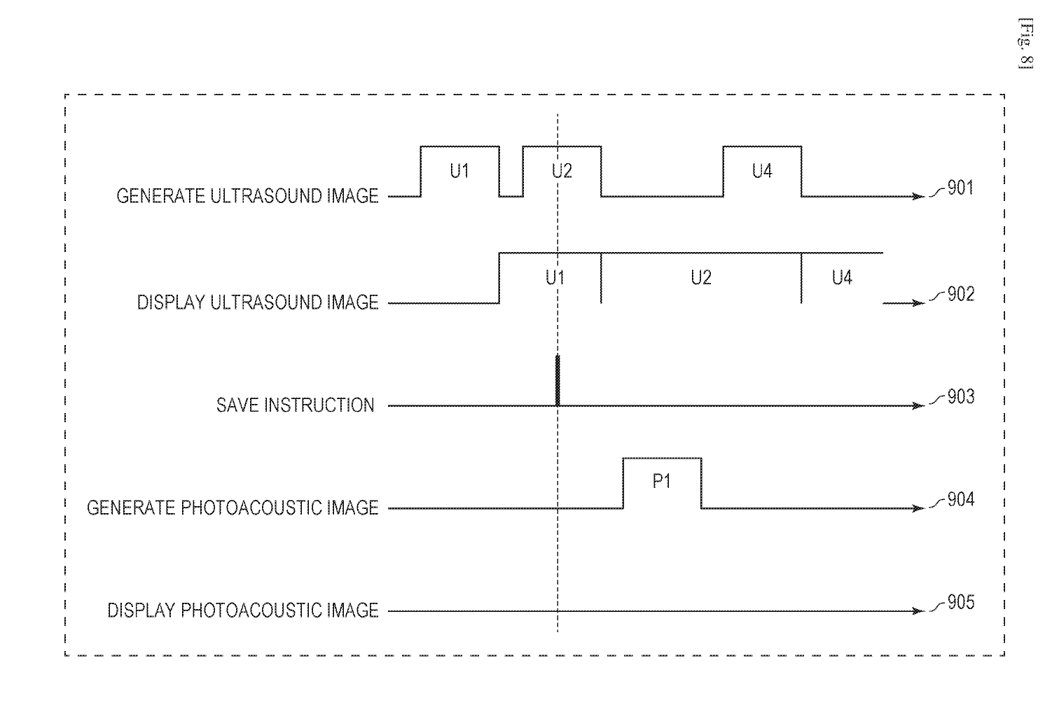

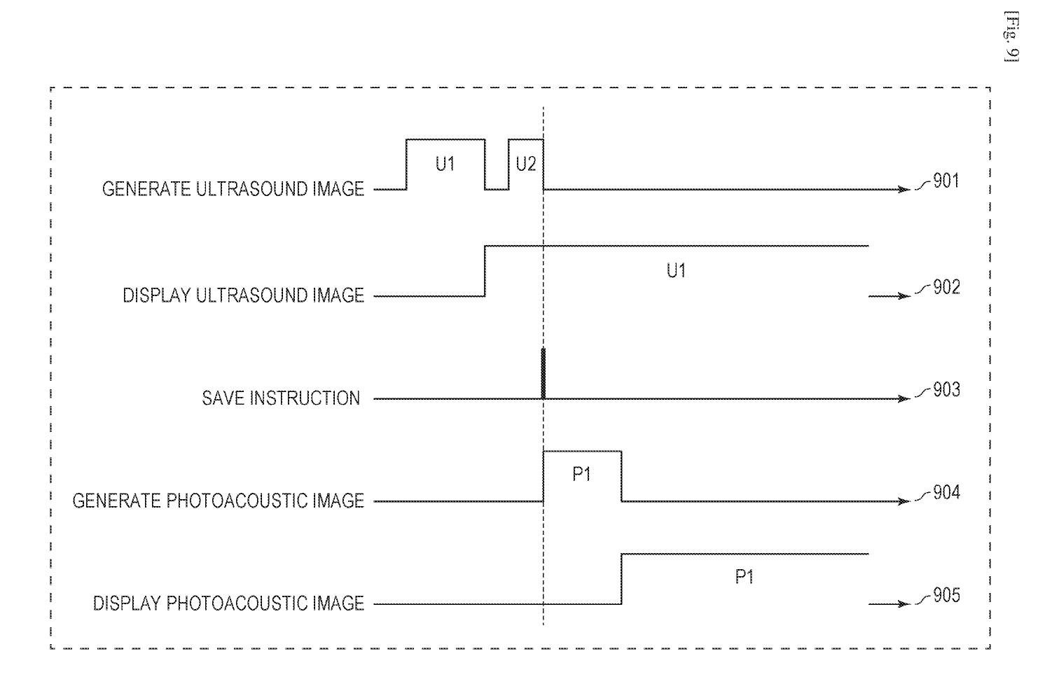

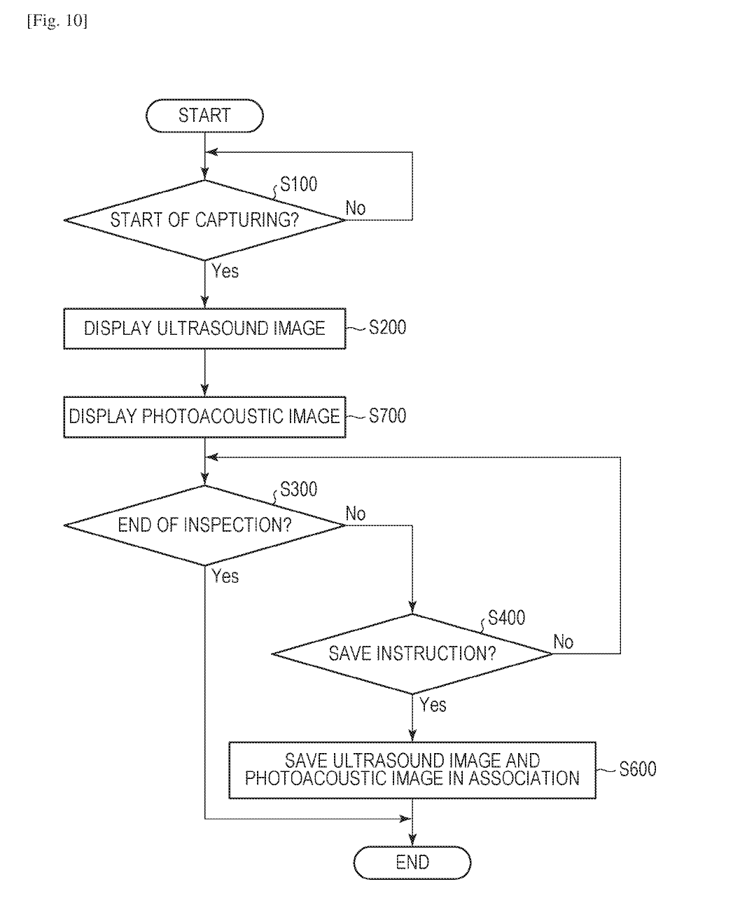

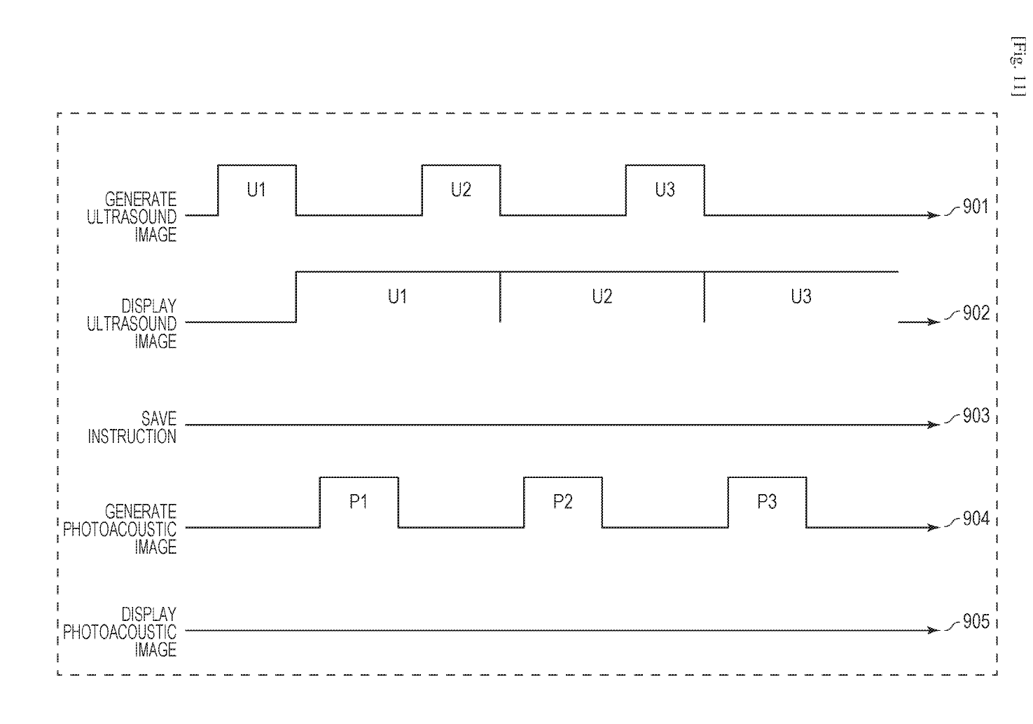

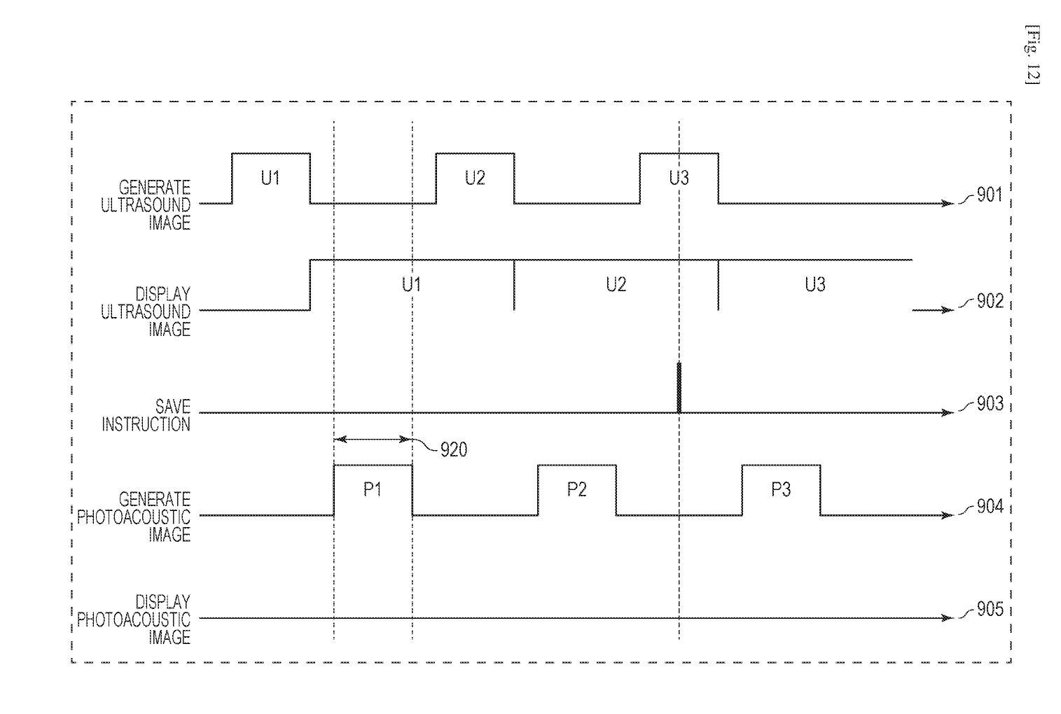

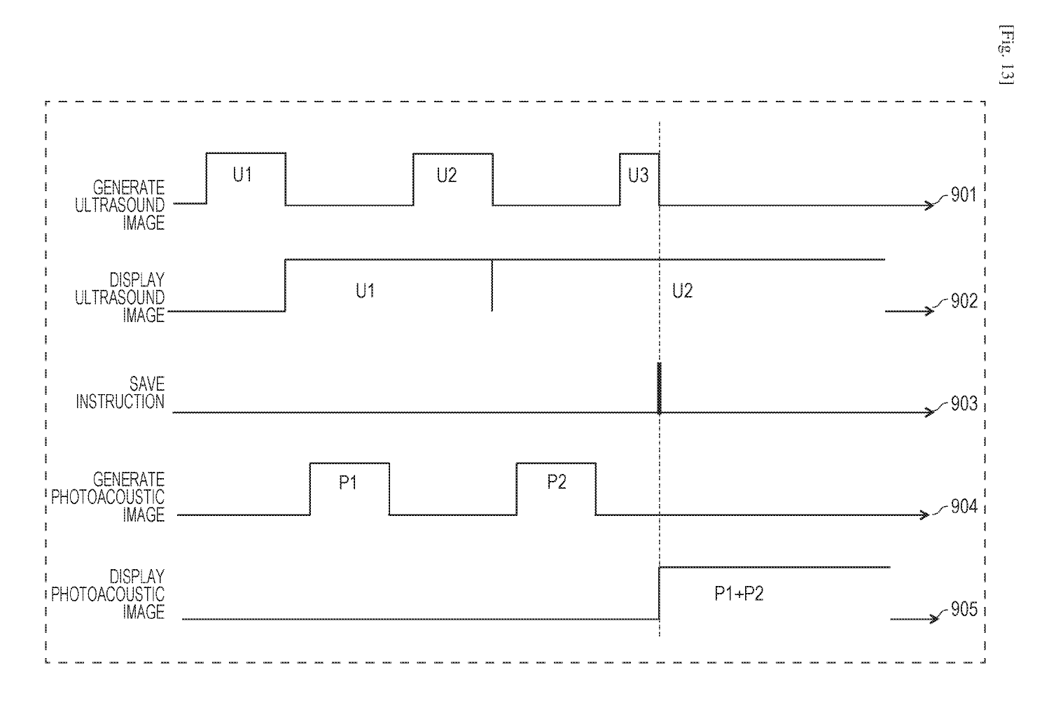

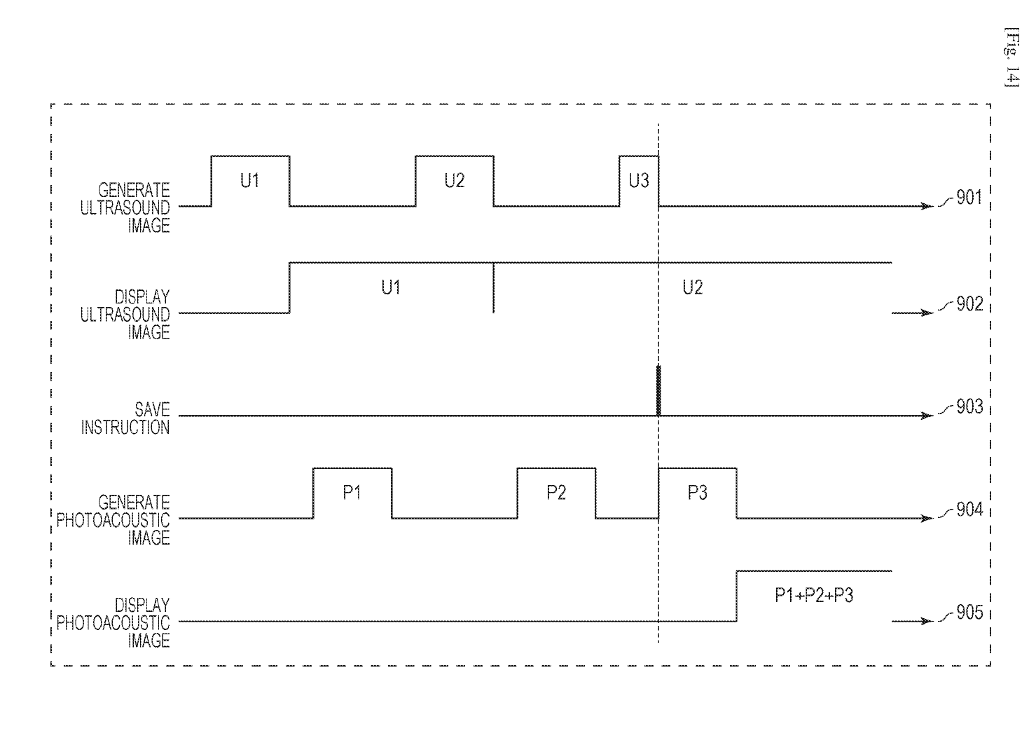

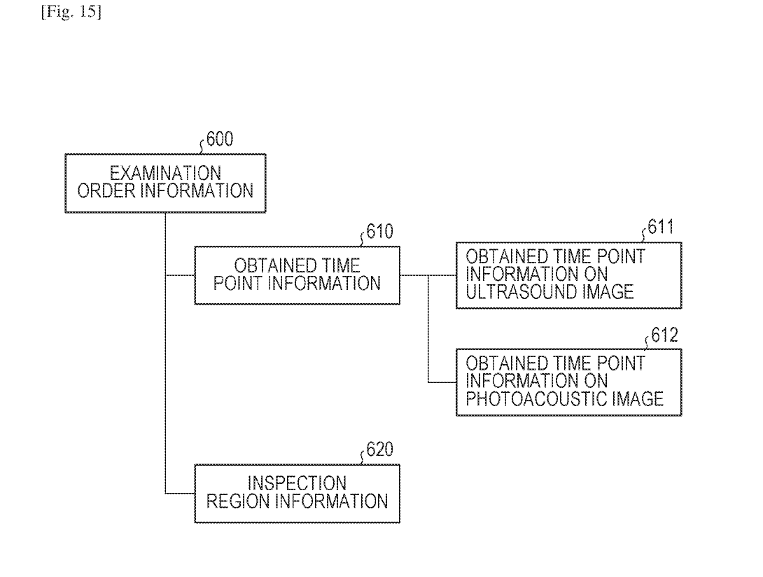

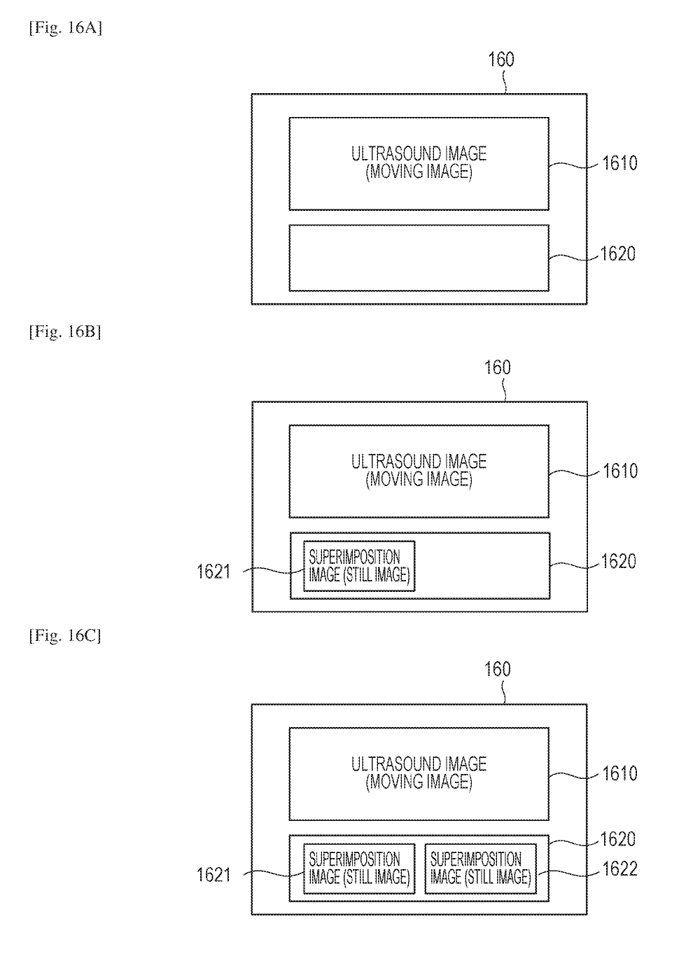

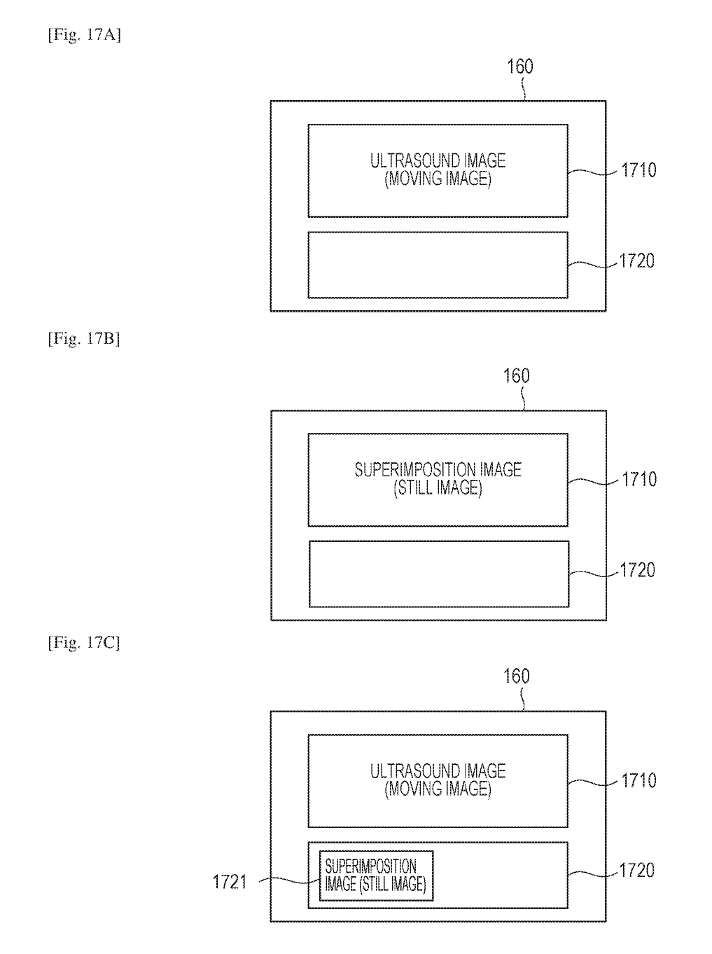

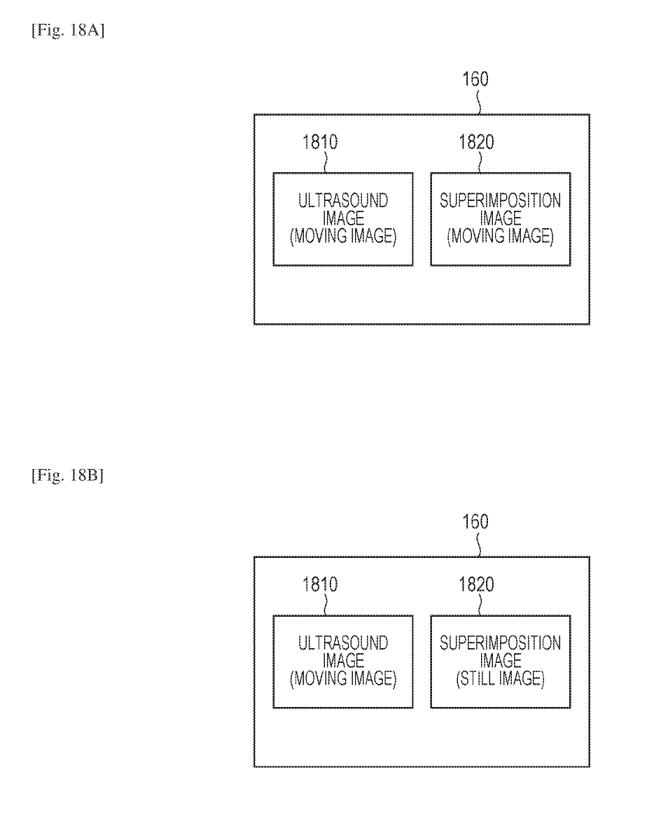

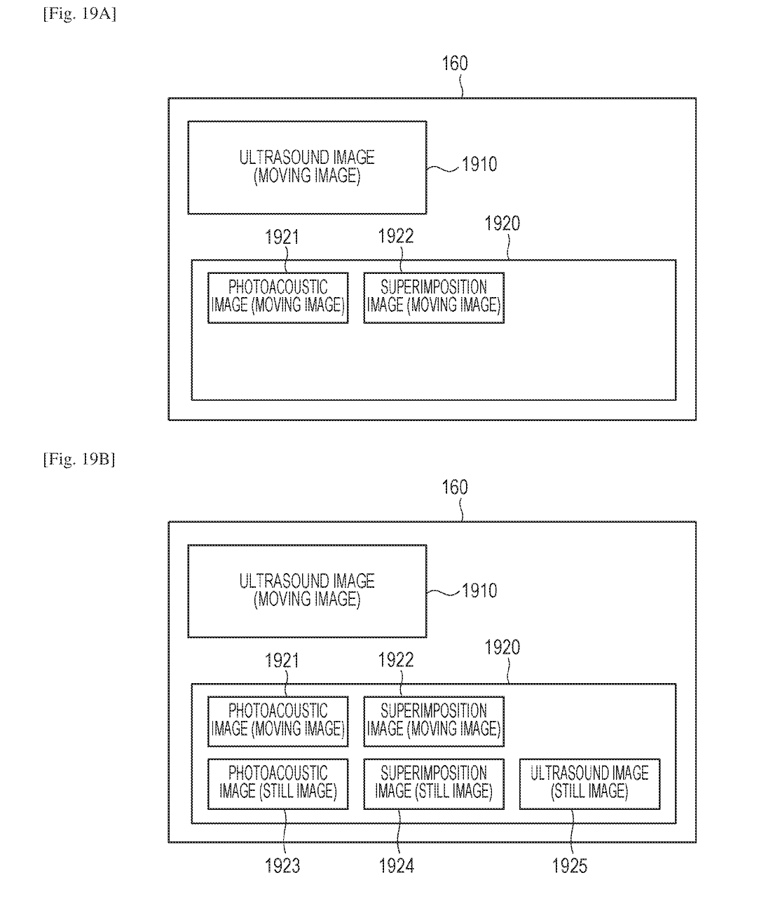

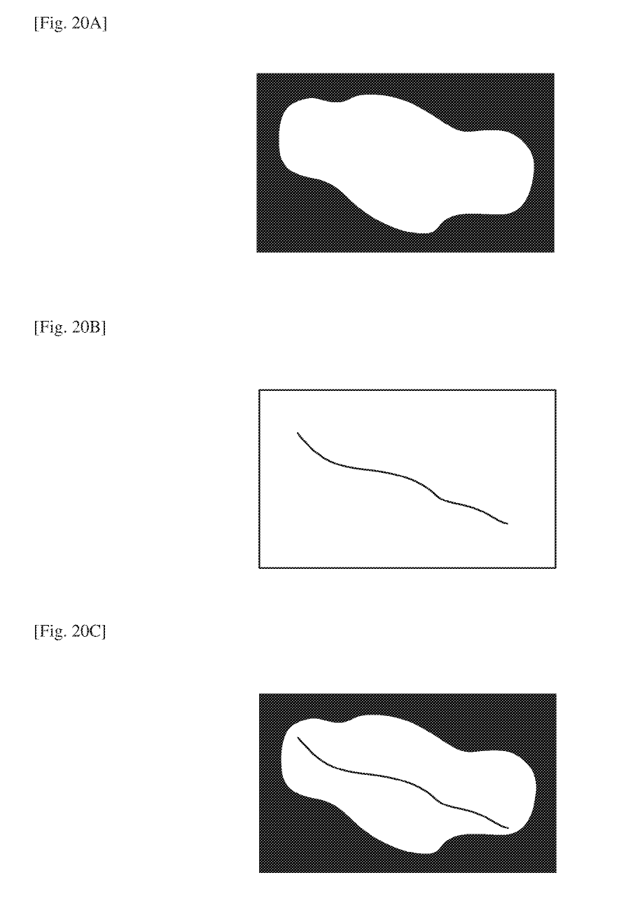

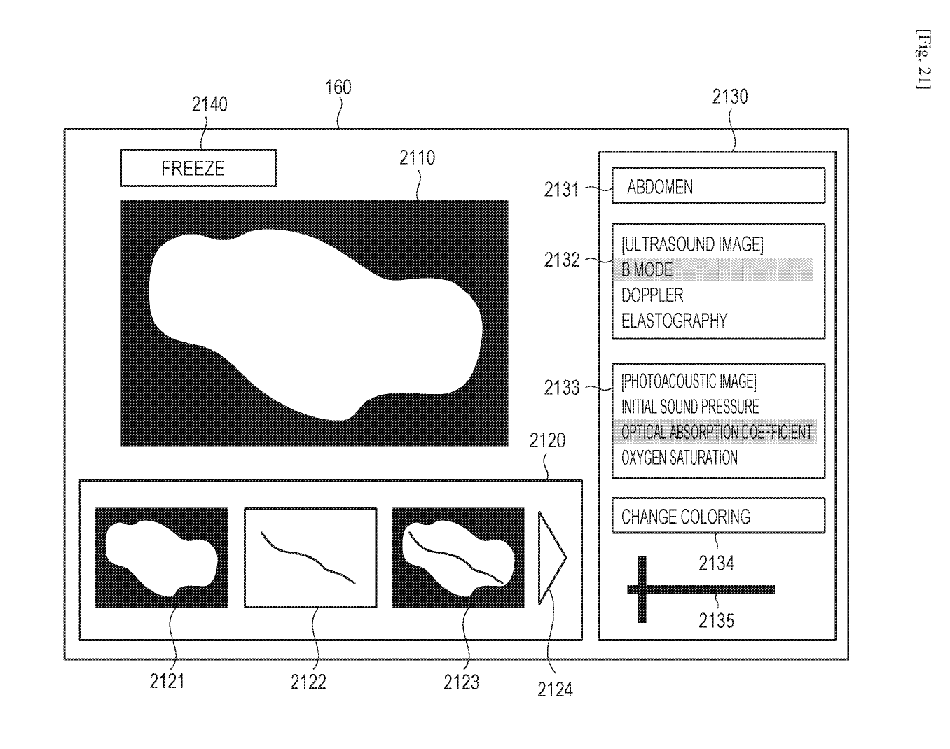

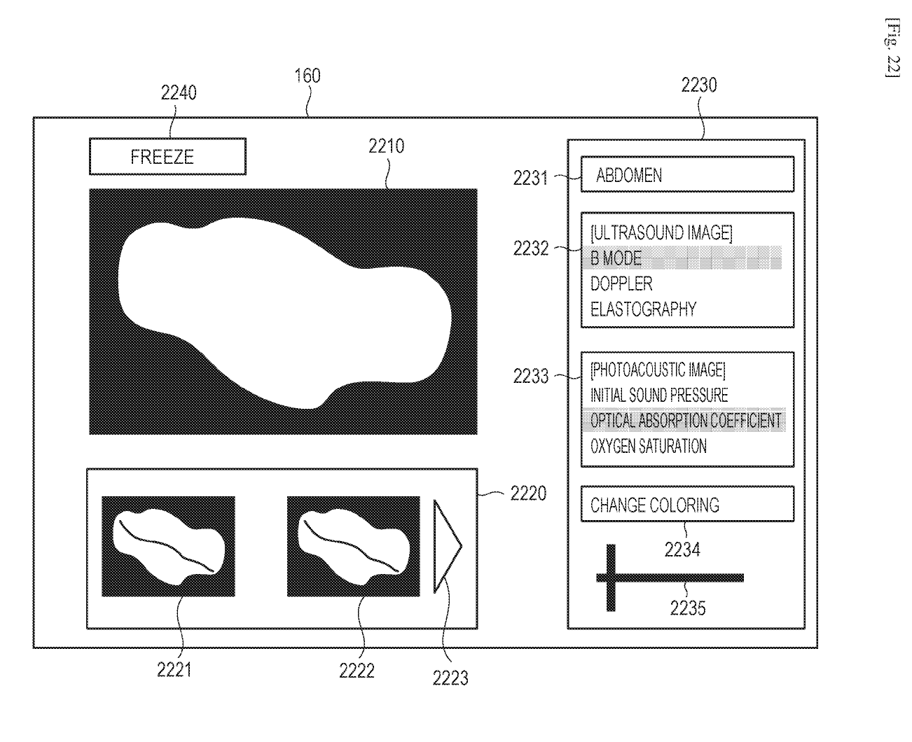

Next, with reference to FIG. 4, a control method for saving a photoacoustic image and an ultrasound image according to this embodiment will be described.

### S100: Determining Whether Start of Capturing is Instructed or Not

The control unit 153 can receive an instruction to start capturing an ultrasound image. If the control unit 153 receives an instruction to start capturing, the processing moves to S200.

When a user instructs to start capturing an ultrasound image by using the input unit 170, the control unit 153 receives information representing the instruction to start capturing (hereinafter, capturing start instruction) from the input unit 170. For example, when a user presses a switch for capturing start provided in the probe 180, the control unit 153 receives information representing the capturing start instruction from the input unit 170.

In this processing, not only an instruction to capture an ultrasound image but also an instruction to capture both of an ultrasound image and a photoacoustic image may be received.

### S200: Displaying Ultrasound Image

The control unit 153 in response to the information representing capturing start performs the following device control.

The probe 180 transmits and receives ultrasonic waves to and from the object 100 to output an ultrasonic signal. The signal data collecting unit 140 performs analog-digital (AD) conversion processing on the ultrasonic signal and transmits the processed ultrasonic signal to the computer 150. The ultrasonic signal being a digital signal is stored in the storage unit 152. The computing unit 151 may perform reconstruction processing including phasing addition (Delay and Sum) on the ultrasonic signal to generate an ultrasound image. When the ultrasound image is generated, the ultrasonic signal saved in the storage unit 152 may be deleted. The control unit 153 being a display control unit transmits the generated ultrasound image to the display unit 160 and performs display control to control the display unit 160 to display the ultrasound image. This processing is repeatedly performed so that the ultrasound image to be displayed by the display unit 160 may be updated. Thus, the ultrasound image can be displayed as a moving image.

In this case, saving all of the ultrasound images displayed as a moving image by the display unit 160 in the storage unit 152 may greatly increase the saved data amount. In order to avoid the problem, when the display image is updated, the previously displayed ultrasound image may be deleted from the storage unit 152. However, in a case where an ultrasound image corresponding to a save instruction, which will be described below, is based on an ultrasound image generated before the save instruction, the ultrasound image may be saved because it is possibly to be saved.

In this processing, a photoacoustic image is not displayed over an ultrasound image. A photoacoustic image may be displayed on the display unit 160 if an ultrasound image can be separately observed. For example, an ultrasound image and a photoacoustic image may be displayed side by side so that the ultrasound image can be separately observed. However, in a case where the display region of the display unit 160 becomes scarce because of display of a photoacoustic image, an ultrasound image may only be displayed without display of a photoacoustic image.

In addition to a display mode in which a photoacoustic image is not superimposed on an ultrasound image as in the aforementioned processing, another display mode may be provided in which an ultrasound image and a photoacoustic image are superimposed to display them as a moving image. In this case, the control unit 153 may be configured to switch the display mode in response to a switching instruction given by a user through the input unit 170. For example, the control unit 153 may be configured to switch between the parallel display mode as a display mode preventing a photoacoustic image from being superimposed on an ultrasound image and the superimposition mode.

### S300: Determining Whether End of Inspection is Instructed or Not

The control unit 153 can receive an instruction to complete an inspection (hereinafter, inspection end instruction). The control unit 153 completes the inspection in response to an inspection end instruction. The control unit 153 can receive the instruction from a user or from an external network such as a hospital information system (HIS) and a radiology information system. The control unit 153 may determine end of an inspection at a time after a lapse of a predetermined time period from the inspection start instruction received in S100.

### S400: Determining Whether Save Instruction is Given or Not

The control unit 153 can receive a save instruction. When the control unit 153 receives a save instruction, the processing moves to S500.

A user may observe ultrasound images displayed as a moving image on the display unit 160 and can give a save instruction by using the input unit 170 when an object to be saved is found among the ultrasound images. In this case, when the display unit 160 displays a still image, a user may instruct to save the image by pressing a freeze button provided in an operating console as the input unit 170, for example. Here, the control unit 153 receives information representing a save instruction from the input unit 170.

The computing unit 151 may perform an image process on the ultrasound image generated in S200 to generate information representing a save instruction if the ultrasound image includes a region of interest and may transmit the information to the control unit 153. For example, when a region of interest is determined based on a user's instruction or an examination order, the computing unit 151 reads out a prestored image pattern corresponding to the region of interest from the storage unit 152 and correlates the image pattern and the ultrasound image generated in S100. The computing unit 151 determines the ultrasound image to be saved if the calculated correlation is higher than a threshold value and generates information representing a save instruction.

The control unit 153 may receive a save instruction from an external network such as an HIS and an RIS.

### S500: Generating Photoacoustic Image

If the control unit 153 receives information representing a save instruction, the control unit 153 may perform the following device controls.

First of all, if the control unit 153 receives information representing a save instruction, the control unit 153 transmits information (control signal) representing light irradiation to the probe 180. The probe 180 having received the information representing light irradiation irradiates light to the object 100, receives photoacoustic waves caused by the light irradiation and outputs a photoacoustic signal. The signal data collecting unit 140 may perform AD conversion processing on the photoacoustic signal and transmit the processed photoacoustic signal to the computer 150. The photoacoustic signal being a digital signal is stored in the storage unit 152. The computing unit 151 may perform reconstruction processing such as Universal Back-Projection (UBP) on the photoacoustic signal to generate a photoacoustic image. Here, a reconstruction region of the photoacoustic image may be an ultrasound image display region displayed when a save instruction is given. In other words, the computing unit 151 may receive information regarding an ultrasound image display region displayed when a save instruction is given and determine a reconstruction region based on the information. When the photoacoustic image is generated, the photoacoustic signal saved in the storage unit 152 may be deleted. However, this is not applicable if the photoacoustic signal is to be used in a process, which will be described below. The inspection system according to this embodiment can be triggered to perform light irradiation by information representing a save instruction to generate a photoacoustic image corresponding to the time point of the save instruction. The probe 180 may perform the light irradiation at a time point of a save instruction or after a lapse of a predetermined time point from a save instruction.

The control unit 153 may control the components to perform light irradiation during a period when it can be determined that there is less influence of a body movement due to breathing or pulsation, instead of in response to a save instruction, to generate a photoacoustic image. For example, the control unit 153 may control the light irradiating unit 110 to perform light irradiation within 250 ms from a save instruction. The control unit 153 may control the light irradiating unit 110 to perform light irradiation within 100 ms from a save instruction. The time period from a save instruction to light irradiation may be equal to a predetermined value or may be designated by a user by using the input unit 170. The control unit 153 may control the time point for light irradiation such that the condition that t1<t2 and |t1−t2| is lower than or equal to α can be satisfied where t1 is a clock time of an ultrasound image save instruction, t2 is a clock time of light irradiation for obtaining a photoacoustic signal, and α is a predetermined value. Alternatively, the control unit 153 may control a time point for light irradiation such that the condition that t1>t2 and |t1−t2| is lower than or equal to α can be satisfied. The predetermined value α may be designated by a user by using the input unit 170.

The control unit 153 may control to perform light irradiation when receiving information describing that it is detected that the probe 180 and the object 100 are brought into contact in addition to information representing a save instruction. This can prevent light irradiation from occurring when the probe 180 and the object 100 are not in contact so that redundant light irradiation can be inhibited.

### S600: Saving Ultrasound Image and Photoacoustic Image in Association

When the control unit 153 as a saving control unit receives information representing a save instruction, the control unit 153 saves an ultrasound image corresponding to the time point of the save instruction and a photoacoustic image generated by being triggered by the save instruction in S500. The photoacoustic image generated by being triggered by a save instruction in S500 corresponds to the photoacoustic image corresponding to the time point of the save instruction. An ultrasound image corresponding to the time point of a save instruction will be described below.

The storage unit 152 may save the ultrasound image displayed on the display unit 160 when a save instruction is received as an ultrasound image corresponding to the time point of the save instruction. The storage unit 152 may save an ultrasound image in a frame neighboring in time to the ultrasound image displayed on the display unit 160 when a save instruction is received as an ultrasound image corresponding to the time point of the save instruction.

An ultrasound image generated during a period when it can be determined that there is less influence of a body movement due to breathing or pulsation, instead of in response to a save instruction, may be saved as an ultrasound image in a frame neighboring in time. For example, the storage unit 152 may save an ultrasound image in a frame less than or equal to ±250 ms from a save instruction as an ultrasound image in a frame neighboring in time. The storage unit 152 may save an ultrasound image in a frame less than or equal to ±100 ms from a save instruction as an ultrasound image in a frame neighboring in time. The target to be saved may be determined with reference to the number of frames. For example, the storage unit 152 may save an ultrasound image less than or equal to ±5 frames from a save instruction as an ultrasound image in a frame neighboring in time. The storage unit 152 may save an ultrasound image within ±1 frame from a save instruction, that is, an adjacent ultrasound image as an ultrasound image in a frame neighboring in time. A time difference or a frame difference between a time point of a save instruction as described above and a time point for obtaining an image to be saved may be equal to a predetermined value or may be designated by a user by using the input unit 170. In other words, a user may use the input unit 170 to designate a range of “neighboring in time”.

Having described that this processing saves an ultrasound image and a photoacoustic image in association, supplementary information may additionally be saved in association with them. For example, in S600, saved data 300 as illustrated in FIG. 5 can be stored in the storage unit 152. The saved data 300 may include supplementary information 310 and image data 320. The image data 320 may include an ultrasound image 321 and a photoacoustic image 322 that are in association with each other. The supplementary information 310 may include object information 311 being information regarding an object 100 and probe information 312 being information regarding the probe 180. The supplementary information 310 includes obtaining time point information 313 being information regarding an obtaining time point (obtaining clock time) of the ultrasound image 321 or the photoacoustic image 322 to be saved in S600.

The object information 311 may include at least one information piece of, for example, object ID, object name, age, blood pressure, heart rate, body temperature, height, weight, medical history, the number of weeks of pregnancy, and an inspection objective region. The inspection system may have an electrocardiographic apparatus or a pulse oximeter (not illustrated) and save information output from the electrocardiographic apparatus or the pulse oximeter at the time point of a save instruction in association therewith as object information. Furthermore, all information regarding an object may be saved as object information.

The probe information 312 includes information regarding the probe 180 such as a position and a gradient of the probe 180. For example, the probe 180 may have a position sensor such as a magnetic sensor, and information regarding an output from the position sensor corresponding to a time point of a save instruction may be saved as the probe information 312.

Information regarding a transmission time point for a control signal for transmission or reception of ultrasonic waves may be saved as the ultrasound image obtaining time point information 313. Information regarding a transmission time point for a control signal for light irradiation may be saved as photoacoustic image obtaining time point information. The inspection system may have a light detecting unit configured to detect pulsed light 113 ejected from the light irradiating unit 110 so that information regarding an output time point of a signal from the light detecting unit can be saved as photoacoustic image obtaining time point information.

Having described with reference to FIG. 5 the saved data 300 including a pair of image data pieces 320 that are associated with each other, a plurality of pairs of image data pieces may be included in one saved data set. In this case, supplementary information regarding a plurality of pairs of image data may also be saved in one saved data set. Alternatively, a plurality of pairs of image data pieces may be saved as different saved data sets. A plurality of image data pieces to be associated may be stored in one data file to associate the plurality of image data pieces. Supplementary information representing which images are to be associated may be attached to image data pieces so that a plurality of image data pieces can be associated.

The saved data may have a data format based on DICOM standard, for example. The format of saved data according to the present invention is not limited to DICOM but may be any data format.

According to this embodiment, in response to a save instruction given when a photoacoustic image is not displayed, an ultrasound image corresponding to the save instruction and a photoacoustic image corresponding to the save instruction may be saved in association. Thus, without switching from ultrasound image display to photoacoustic image display, the photoacoustic image can be saved. This can reduce the time lag from confirmation of a region of interest in the ultrasound image to saving the photoacoustic image. Because, according to this embodiment, light irradiation is triggered by a save instruction, redundant light irradiation can be inhibited. This further can suppress power consumption due to redundant light irradiation.

According to this embodiment, a photoacoustic image is saved in association with an ultrasound image. However, without limiting to a photoacoustic image as information representing a spatial distribution, information derived from a photoacoustic signal can be saved in association therewith. For example, a photoacoustic signal (RAW data) itself, an average concentration of a substance contained in an object, a pixel value at a specific position in a spatial distribution, or a statistic value (such as an average value or a median value) of pixel values in the spatial distribution may be associated with an ultrasound image as information derived from a photoacoustic signal.

After an ultrasound image and a photoacoustic image are associated based on a save instruction, the associated images may be superimposed for display on the display unit 160. The display of the resulting superimposition image may be triggered by a save instruction or may be executed based on an instruction from a user.

Next, with reference to FIGS. 6 to 8, a measurement sequence according to this embodiment will be described. Each of diagrams 901 to 905 has a time axis horizontally where time passes as it goes to the right.

The diagram 901 illustrates timing for generating an ultrasound image. Transmission of ultrasonic waves starts at rises in the diagram 901, and generation of an ultrasound image completes at drops in the diagram 901. The diagram 902 illustrates ultrasound image display timing. When generation of an ultrasound image completes, display of the ultrasound image is enabled. The processing in S200 corresponds to the diagrams 901 and 902.

The diagram 903 illustrates timing of a save instruction. A rise in the diagram 903 indicates a time point when a save instruction is received. The processing in S400 corresponds to the diagram 903.

The diagram 904 illustrates timing for generating a photoacoustic image. Light irradiation starts at a rise in the diagram 904, and generation of a photoacoustic image completes at a drop in the diagram 904. The processing in S500 corresponds to the diagram 904.

The diagram 905 illustrates timing for displaying a photoacoustic image. When generation of a photoacoustic image completes, display of the photoacoustic image is enabled.

FIG. 6 is a timing chart where no save instruction is given. When no save instruction is given, ultrasonic waves are transmitted and are received, and, when generation of an ultrasound image completes, processing of updating the displayed ultrasound image is repeated. In other words, ultrasound images U1, U2, U3, and U4 are displayed in order of ultrasound images U1, U2, U3, and U4 as a moving image. In this case, neither light irradiation nor photoacoustic image generation is performed.

FIG. 7 is a timing chart where a save instruction is received when the ultrasound image U1 is being displayed. In this case, generation of the ultrasound image U2 is discontinued after a save instruction is received, and generation of a photoacoustic image P1 starts. After the generation of the photoacoustic image P1 completes, the ultrasound image U1 and the photoacoustic image P1 are saved in association. Thus, the photoacoustic image P1 can be generated without a long time from generation of the ultrasound image U1 to be saved, and the ultrasound image U1 and the photoacoustic image P1 can be saved in association.

As described above, an ultrasound image corresponding to the time point of a save instruction may be saved in association with an ultrasound image other than the ultrasound image U1. This is also true in the following case.

Light may be irradiated a plurality of number of times during a period 910 for generation of a photoacoustic image P1 having a high S/N, and the photoacoustic signals corresponding to the plurality of number of times of light irradiation may be used to generate the photoacoustic image P1. This is also true in the following case. Because the generation of the ultrasound image U2 is discontinued halfway, the display of the ultrasound image U1 may be continued when the photoacoustic image P1 is being generated and when the ultrasound image U3 is being generated.

FIG. 8 is another timing chart in a case where a save instruction is received when the ultrasound image U1 is being displayed. Unlike FIG. 7, the generation of the ultrasound image U2 is continued when the save instruction is received, instead of discontinuing the generation of the ultrasound image U2. After the generation of the ultrasound image U2 completes, the generation of the photoacoustic image P1 starts. Also in this case, the photoacoustic image P1 and the ultrasound image U1 can be saved in association. The ultrasound image U2 being an ultrasound image corresponding to the time point of the save instruction and the photoacoustic image P1 may be saved in association. In this case, images that are closer in time can be saved in association, compared with saving in association with the ultrasound image U1.

FIG. 9 is another timing chart in a case where a save instruction is received when the ultrasound image U1 is being displayed. In this case, when the save instruction is received, the ultrasound image U1 and photoacoustic image P1 are superimposed to display a still image. More specifically, when the save instruction is received, the display of the ultrasound image P1 is continued and, at the same time, the save instruction triggers to start the generation of the photoacoustic image P1. When the generation of the photoacoustic image P1 completes, the ultrasound image U1 and the photoacoustic image P1 are saved in association with each other, and the photoacoustic image P1 in association with the ultrasound image U1 being displayed is superimposed thereon for display. Thus, a user can perform diagnosis by watching the still image having the ultrasound image U1 and photoacoustic image P1 that are saved in association.

In a case where a save instruction is given in a display mode in which an ultrasound image and a photoacoustic image are superimposed for display as a moving image as described with reference to S300, the ultrasound image and photoacoustic image displayed on the display unit 160 when the save instruction is given may be saved in association with each other.

### Second Embodiment

According to the first embodiment, a save instruction triggers to start light irradiation and generation of a photoacoustic image, and the resulting photoacoustic image is saved in association with an ultrasound image. On the other hand, according to a second embodiment, a photoacoustic image which is obtained based on a photoacoustic image generated at a predetermined time point and which corresponds to the time point of the save instruction is saved in association with an ultrasound image.

This embodiment will also be described with reference to the inspection system according to the first embodiment. Like numbers refer to like parts in principle, and any repetitive description will be omitted.

With reference to a flowchart illustrated in FIG. 10, a method for saving an ultrasound image and a photoacoustic image according to the second embodiment will be described. Like numbers refer to like steps, and any repetitive description will be omitted.

### S700: Generating Photoacoustic Image

An inspection system according to this embodiment, light irradiation is performed at a predetermined time point, and a photoacoustic signal is obtained so that the photoacoustic signal can be used to generate a photoacoustic image. For example, the inspection system performs light irradiation in accordance with a repetition frequency of a light source and generates a photoacoustic image at the repetition frequency. The inspection system may further generate one photoacoustic image by performing light irradiation a plurality of number of times.

In order to prevent increases of the saved data amount, the storage unit 152 may save one photoacoustic image only. In other words, every time a new photoacoustic image is generated, the photoacoustic image to be saved in the storage unit 152 is updated therewith. The lastly saved photoacoustic image may be deleted from the storage unit 152. However, in a case where a photoacoustic image corresponding to a time point of a save instruction, which will be described below, is based on a photoacoustic image generated before the time point of the save instruction, the photoacoustic image may be saved because it is possibly to be saved. When the ultrasound image to be displayed on the display unit 160 is updated, the photoacoustic image to be saved in the storage unit 152 may be updated.

When a photoacoustic image is generated, the photoacoustic signal saved in the storage unit 152 may be deleted. However, the deletion may be performed except in cases where the photoacoustic signal is to be used in a process, which will be described below.

The processing in S700 may be performed before the processing in S200. Also in this case, the superimposition of a photoacoustic image on an ultrasound image is not performed in S200.

This processing may obtain information derived from a photoacoustic signal, without limiting to a photoacoustic image functioning as information representing a spatial distribution of object information. In other words, this processing may generate a photoacoustic image functioning as information representing a spatial distribution of object information. For example, a photoacoustic signal (RAW data) itself, an average concentration of a substance contained in an object, a pixel value at a specific position in a spatial distribution, or a statistic value (such as an average value or a median value) of pixel values in the spatial distribution may be obtained as information derived from a photoacoustic signal. The time point for obtaining a photoacoustic image corresponds to the light irradiation time point for obtaining the photoacoustic signal. It is assumed hereinafter that saving a photoacoustic image includes saving information derived from a photoacoustic signal.

### S900: Saving Ultrasound Image and Photoacoustic Image in Association

When the control unit 153 according to this embodiment receives information representing a save instruction, the control unit 153 saves an ultrasound image and a photoacoustic image corresponding to the time point of the save instruction in association. The same processing as that of the first embodiment is performed on an ultrasound image corresponding to the time point of a save instruction. The photoacoustic image corresponding to the time point of the save instruction will be described below.

According to this embodiment, the control unit 153 obtains a photoacoustic image corresponding to the time point of a save instruction based on a photoacoustic image neighboring in time to the time point of the save instruction among photoacoustic images generated in S700. For example, the control unit 153 may use a photoacoustic image generated during a period when it can be determined that there is less influence of a body movement due to breathing or pulsation in response to a save instruction as a photoacoustic image in a frame neighboring in time. For example, the storage unit 152 may save a photoacoustic image in a frame within ±250 ms from a save instruction as a photoacoustic image in a frame neighboring in time. The storage unit 152 may save a photoacoustic image in a frame within ±100 ms from a save instruction as a photoacoustic image in a frame neighboring in time. A photoacoustic image to be saved may be determined with reference to the number of frames. For example, the storage unit 152 may save a photoacoustic image within ±5 frames from a save instruction as a photoacoustic image in a frame neighboring in time. The storage unit 152 may save a photoacoustic image within ±1 frame from or a photoacoustic image adjacent to a save instruction as a photoacoustic image in a frame neighboring in time. A time difference or a frame difference between a time point of a save instruction as described above and a time point for obtaining an image to be saved may be a predetermined value or may be designated by a user by using the input unit 170. In other words, a user may use the input unit 170 to designate a range of “neighboring in time”. The control unit 153 may determine a photoacoustic image to be saved such that the condition that t1<t2 and |t1−t2| is lower than or equal to α can be satisfied where t1 is a clock time of an image save instruction, t2 is a clock time of a time point for obtaining an photoacoustic image to be saved, and α is a predetermined value. Alternatively, the control unit 153 may determine a photoacoustic image to be saved such that the condition that t1>t2 and |t1−t2| is lower than or equal to α can be satisfied. The predetermined value α may be designated by a user by using the input unit 170.

Photoacoustic images in a plurality of frames neighboring in time may be synthesized to obtain a photoacoustic image to be saved in association. The control unit 153 can obtain a photoacoustic image to be saved by synthesizing photoacoustic images in a plurality of frames by simple addition, addition average, weighting addition, or weighting addition average, for example. Some types of the synthesizing processing may be performed by the computing unit 151 as in other types of processing.

This processing may obtain not only a photoacoustic image to be saved but also information derived from a photoacoustic signal corresponding to the time point of a save instruction. The ultrasound image corresponding to the time point of the save instruction and the information derived from the photoacoustic signal corresponding to the time point of the save instruction may then be saved in association with each other. The computing unit 151 may synthesize information pieces derived from photoacoustic signals corresponding to a plurality of number of times of light irradiation to generate synthesized information, like the synthesizing processing.

The present invention may not save a photoacoustic image and an ultrasound image that are associated with each other in a storage unit in the inspection system. The control unit may save a photoacoustic image and an ultrasound image that are associated with each other in an image management system such as a PACS (Picture Archiving and Communication System) connected to an external network.

Next, with reference to FIGS. 11 to 14, a measurement sequence according to this embodiment will be described. A diagram 901 illustrates timing for generating an ultrasound image. A diagram 902 illustrates ultrasound image display timing. When generation of an ultrasound image completes, display of the ultrasound image is enabled. A diagram 903 illustrates timing of a save instruction. A diagram 904 illustrates timing for generating a photoacoustic image. The processing in S700 corresponds to the diagram 904. A diagram 905 illustrates timing for displaying a photoacoustic image. When generation of a photoacoustic image completes, display of the photoacoustic image is enabled.

FIG. 11 is a timing chart where no save instruction is given. When no save instruction is given, ultrasonic waves are transmitted and are received, and, when generation of an ultrasound image completes, processing of updating the displayed ultrasound image is repeated. In other words, ultrasound images U1, U2, U3, and U4 are displayed in order of ultrasound images U1, U2, U3, and U4 as a moving image. On the other hand, a photoacoustic image is generated between generations of an ultrasound image. In other words, generation of an ultrasound image and generation of a photoacoustic image are executed alternately. In this case, a photoacoustic image is generated, but the photoacoustic image is not saved and displayed.

FIG. 12 is a timing chart in a case where a save instruction is received when the ultrasound image U2 is being displayed. In this case, the ultrasound image U2 displayed when a save instruction is received and the photoacoustic image P1 or the photoacoustic image P2 adjacent in time to the ultrasound image U2 can be saved in association. A photoacoustic image corresponding to light irradiation closer in time to transmission and reception of ultrasonic waves for generation of the ultrasound image U2 may be saved in association with the ultrasound image U2. Alternatively, a composition image of the photoacoustic image P1 and the photoacoustic image P2 and the ultrasound image U1 may be saved in association.

The ultrasound image U1 or the ultrasound image U3 neighboring in time to the ultrasound image U2 may be saved. In this case, a photoacoustic image neighboring in time to the ultrasound image U1 or the ultrasound image U3 may be saved.

Having described that the photoacoustic image P1 is generated during the period 920, a photoacoustic signal may only be obtained during the period 920 without generation of the photoacoustic image P1. In this case, the computing unit 151 may use a photoacoustic signal obtained during the period 920 after a save instruction is received to generate the photoacoustic image P1 and save the photoacoustic image P1 in association with the ultrasound image U2. During the period 920, instead of the photoacoustic image P1, information derived from a photoacoustic signal may be generated and be saved in association with the ultrasound image U2. These are also true for other photoacoustic images.

FIG. 13 is another timing chart in a case where a save instruction is received when the ultrasound image U2 is being displayed. In this case, when a save instruction is received, the ultrasound image U2 and a photoacoustic image P1+P2 being a composition image of the photoacoustic image P1 and the photoacoustic image P2.

Furthermore, in this case, a still image of the ultrasound image U2 displayed upon reception of a save instruction is continuously displayed. The generation of the ultrasound image U3 is discontinued. In other words, a save instruction triggers to switch from moving image display to still image display. Furthermore, in this case, a still image of the photoacoustic image P1+P2 saved in association with the ultrasound image U2 is superimposed on the still image of the ultrasound image U2 for display.

FIG. 14 is another timing chart in a case where a save instruction is received when the ultrasound image U2 is being displayed. In this case, the still image display of the ultrasound image U2 is continued when a save instruction is received. When a save instruction is received, the generation of the ultrasound image U3 is discontinued, and generation of the photoacoustic image P3 is started. When the generation of the photoacoustic image P3 completes, the ultrasound image U2 and a photoacoustic image P1+P2+P3 are saved in association. Then, the photoacoustic image P1+P2+P3 associated with the ultrasound image U2 is superimposed on the currently displayed ultrasound image U2 for display. Here, the photoacoustic image P1+P2+P3 is a composition image of the photoacoustic image P1, the photoacoustic image P2, and the photoacoustic image P3.

In the case illustrated in FIG. 14, the S/N ratio of a photoacoustic image can be improved more than the case in FIG. 13. Because a save instruction triggers to discontinue transmission and reception of ultrasonic waves for prioritizing reception of photoacoustic waves, the time interval from obtaining of the ultrasound image U2 to obtaining of the photoacoustic image P3 can be reduced.

### Third Embodiment

An inspection system according to a third embodiment determines images to be saved in association with each other based on examination order information transmitted from an external network such as an HIS or an RIS. FIG. 15 illustrates a data structure of examination order information 600 obtained by the inspection system according to this embodiment.

Information included in the examination order information 600 is directly input by a doctor, for example, by using an HIS or an RIS. Alternatively, an HIS or an RIS, for example, may generate information to be included in the examination order information 600 based on information input by a doctor, for example.

The examination order information 600 includes obtaining time point information 610. The obtaining time point information 610 is information representing at which time point an ultrasound image or a photoacoustic image is to be obtained with reference to the time point of a save instruction. The obtaining time point information 610 includes ultrasound image obtaining time point information 611 and photoacoustic image obtaining time point information 612. For example, the obtaining time point information 610 corresponds to information representing a relationship between a save instruction and an ultrasound image or a photoacoustic image to be saved as in the first or second embodiment.

The control unit 153 reads out the ultrasound image obtaining time point information 611 from the examination order information 600. The control unit 153 when receiving information representing a save instruction sets an ultrasound image obtaining time point corresponding to the time point of the save instruction based on the ultrasound image obtaining time point information 611. The control unit 153 determines an ultrasound image obtained at the set obtaining time point to be saved.

The control unit 153 reads out the photoacoustic image obtaining time point information 612 from the examination order information 600. The control unit 153 when receiving information representing a save instruction sets a photoacoustic image obtaining time point corresponding to the time point of the save instruction based on the photoacoustic image obtaining time point information 612. According to the first embodiment, the control unit 153 controls the probe 180 to irradiate light to the object 100 at the set obtaining time point. Then, the photoacoustic image obtained due to the light irradiation is determined to be saved. On the other hand, according to the second embodiment, the control unit 153 determines a photoacoustic image obtained at the set obtaining time point to be saved.

Thus, an ultrasound image and a photoacoustic image obtained based on the obtaining time point information 610 included in the examination order information 600 are stored in the storage unit 152. The obtaining time point information 610 read from the examination order information 600 is saved as the obtaining time point information 313 for the saved data 300.

The examination order information 600 may include inspection region information 620 which is information regarding a region to be inspected such as the head and the breast. The control unit 153 may read out the inspection region information 620 from the examination order information 600 and may set a predetermined ultrasound image or photoacoustic image obtaining time point for each inspection region based on the inspection region information 620. In this case, when the obtaining time point information 610 is not included in the examination order information 600, the control unit 153 can set an ultrasound image or photoacoustic image obtaining time point based on the examination order information 600. For example, the control unit 153 can read out an obtaining time point corresponding to an inspection region with reference to a relationship table describing correspondence between inspection region and obtaining time point, which is stored in the storage unit 152. The control unit 153 may obtain an obtaining time point based on any information included in examination order information, instead of the information regarding an inspection region, if the information is associated with an obtaining time point.

The control unit 153 can set a type of photoacoustic image to be generated based on the inspection region information 620, such as an oxygen saturation distribution set as the type of photoacoustic image to be generated, based on inspection region information 620 attached to the examination order information 600.

For example, the examination order information 600 may include information regarding the type of ultrasound image or photoacoustic image to be captured and the type of contrast agent to be used instead of the obtaining time point information 610. Additionally or alternatively, the examination order information 600 may include information regarding the type of probe for capturing an ultrasound image or a photoacoustic image, the position of the probe, an output to the probe such as voltage, and the sex, age, physical size, medical history, the number of weeks of pregnancy, and body temperature of an object.

The control unit 153 may compare saved data regarding a previously inspected object and the examination order information 600 and, for example, if the objects therein are matched, set an obtaining time point based on a previous inspection result.

### Fourth Embodiment

Display examples of images to be displayed in the display unit 160 according to a fourth embodiment will be described. A photoacoustic image according to this embodiment is a concept including an image derived from photoacoustic waves generated from irradiated light, as described above. In other words, the photoacoustic image is not limited to an image representing a spatial distribution but may be an image representing a numerical value or a character. The display control, which will be described below, is executed by the control unit 153 being a display control unit.

### Display Form 1

FIGS. 16A to 16C illustrate forms displaying, in response to a save instruction given when a moving image of an ultrasound image is displayed, thumbnails of superimposition images of an ultrasound image and a photoacoustic image corresponding to the time point of the save instruction.

First of all, referring to FIG. 16A, a moving image of an ultrasound image is displayed on a first display area 1610 in the display unit 160 (S200). In other words, simultaneously with the transmission and reception of ultrasonic waves to the object 100 by the probe 180, a moving image of an ultrasound image corresponding to the transmission and reception of the ultrasonic waves is displayed on the first display area 1610.

In response to a save instruction when the moving image of the ultrasound image is displayed (S400), a still image 1621 of a superimposition image of the ultrasound image and the photoacoustic image is displayed on a second display area 1620 in the display unit 160, as illustrated in FIG. 16B. Here, the still image 1621 displayed on the second display area 1620 is a still image of a superimposition image of an ultrasound image and a photoacoustic image corresponding to the time point when the save instruction is given in S400. The still image 1621 being a thumbnail can be displayed in a smaller size than that of the moving image of the ultrasound image displayed on the first display area 1610. The ultrasound image and photoacoustic image corresponding to the time point of the save instruction correspond to the images to be saved in association with S600 or S900.

When the next save instruction is given, a still image 1622 of a superimposition image of an ultrasound image and a photoacoustic image corresponding to the save instruction is displayed on the second display area 1620, as illustrated in FIG. 16C. Thus, superimposition images corresponding to time points of a plurality of save instructions are horizontally displayed on the second display area 1620.

This display form enables a moving image of ultrasound images to be displayed continuously without large occupation of the display range of a photoacoustic image in the whole display area. This can suppress hindrance against diagnosis with a moving image of an ultrasound image and allows auxiliary diagnosis with a photoacoustic image. Having described that, according to this embodiment, a still image of a superimposition image is displayed in response to a still image display instruction, a still image of a photoacoustic image may be displayed instead of a superimposition image. This is also true for the following display forms.

### Display Form 2

FIGS. 17A to 17C illustrate forms each displaying a still image of a superimposition image for a predetermined period of time instead of a moving image of an ultrasound image in response to a save instruction given when a moving image of an ultrasound image is displayed.

First of all, referring to FIG. 17A, a moving image of an ultrasound image is displayed on a first display area 1710 in the display unit 160 (S200).

In response to a save instruction when the moving image of the ultrasound image is displayed (S400), the image displayed on the first display area 1710 is changed to a still image of a superimposition image corresponding to the save instruction, as illustrated in FIG. 17B. Then, the still image of the superimposition image is displayed on the first display area 1710 for a predetermined period of time from the time point of the save instruction. Because it is assumed that a moving image of an ultrasound image is basically displayed for diagnosis, a still image of a superimposition image on the first display area 1710 may be limitedly displayed for the predetermined period of time.

In other words, after a lapse of the predetermined period of time from the save instruction, the image displayed on the first display area 1710 is changed to a moving image of the ultrasound image as illustrated in FIG. 17C while a still image 1721 of the superimposition image corresponding to the save instruction is displayed on the second display area 1720. In other words, after a lapse of the predetermined period of time from the save instruction, the display of a moving image of an ultrasound image on the first display area 1710 is restarted while the still image 1721 of a superimposition image display on the first display area 1710 is displayed as a thumbnail on the second display area 1720. The still image 1721 as a thumbnail may be displayed in a smaller size than that of the moving image of the ultrasound image displayed on the first display area 1710.

The predetermined period of time for displaying a still image of a superimposition image on the first display area 1710 may be a time period which does not hinder the display of the moving image of the ultrasound image while providing a time for checking the still image of the superimposition image. For example, the predetermined period of time may be a time shorter than or equal to two seconds. In a case where priority is given to display of a moving image of an ultrasound image, the predetermined period of time may be a time shorter than or equal to one second or 0.5 seconds.

Alternatively, when a save instruction is given, a still image of a photoacoustic image may be displayed on the first display area 1710, instead of a still image of a superimposition image. Then, a still image of a photoacoustic image or a superimposition image may be displayed on the second display area 1720 after a lapse of a predetermined period of time from the time point of the save instruction.

According to this display form, because a photoacoustic image occupies a large part of the display area only for a predetermined limited period of time, a moving image of an ultrasound image can be displayed continuously. This can suppress hindrance against diagnosis with a moving image of an ultrasound image and allows auxiliary diagnosis with a photoacoustic image.

### Display Form 3

FIGS. 18A and 18B illustrate forms each displaying a moving image of an ultrasound image and a moving image of a superimposition image on different display areas and changing the display of the superimposition image to the display of a still image in response to a save instruction while displaying the moving image of the ultrasound image.

First of all, referring to FIG. 18A, a moving image of an ultrasound image is displayed on a first display area 1810 in the display unit 160 (S200). Furthermore, as illustrated in FIG. 18A, a moving image of a superimposition image of an ultrasound image and a photoacoustic image is displayed on a second display area 1820 in the display unit 160. For example, the ultrasonic wave transmission and reception or light irradiation is performed at time points as illustrated in FIGS. 11 to 14 so that moving images of an ultrasound image and a photoacoustic image are generated to update the images sequentially.

In response to a save instruction given when a moving image of an ultrasound image is displayed (S400), the display as illustrated in FIG. 18B is provided. In other words, in response to a save instruction, a still image of a superimposition image of an ultrasound image and a photoacoustic image is displayed on a second display area 1820 while the display of the moving image of the ultrasound image is continued on the first display area 1810. Here, the still image of the superimposition image is a still image of a superimposition image of an ultrasound image and a photoacoustic image corresponding to the time point of the save instruction. Alternatively, in response to a save instruction, the display of the moving image of the superimposition image on the second display area 1820 may be continued when the still image of the superimposition image may be displayed on a third display area different from the second display area.

The second display area 1820 may display a moving image or a still image of a photoacoustic image instead of a moving image or a still image of the superimposition image.

According to this display form, display of a moving image or a still image of a photoacoustic image can be controlled in response to a save instruction independently from a moving image of an ultrasound image. This can suppress hindrance against diagnosis with a moving image of an ultrasound image and allows auxiliary diagnosis with a photoacoustic image.

### Display Form 4

FIGS. 19A and 19B illustrate a form displaying a moving image of a photoacoustic image and a moving image of a superimposition image in addition to display of a moving image of an ultrasound image and, in response to a save instruction, and displaying still images of a photoacoustic image, an ultrasound image, and a superimposition image on a display area different from the display area for the moving images.

First, referring to FIG. 19A, a moving image of an ultrasound image is displayed on a first display area 1910 in the display unit 160 (S200). Referring to FIG. 19A, a moving image 1921 of a photoacoustic image and a moving image 1922 of a superimposition image of an ultrasound image and a photoacoustic image are displayed on a second display area 1920 in the display unit 160.

When a save instruction is given (S400) when a moving image of an ultrasound image is being displayed, images are displayed as illustrated in FIG. 19B. In other words, in response to a save instruction, the second display area 1920 displays a still image 1923 of a photoacoustic image, a still image 1924 of a superimposition image, and a still image 1925 of an ultrasound image, in addition to the moving images 1921 and 1922. Here, the still image 1924 is a still image of a superimposition image of an ultrasound image and a photoacoustic image corresponding to a time point of a save instruction. For easy comparison between a moving image and a still image that are corresponding to each other, the moving image 1921 and the still image 1923 of a photoacoustic image may be displayed horizontally, and the moving image 1922 and the still image 1924 of a superimposition image may be displayed horizontally. Referring to FIG. 19B, a moving image and a still image may be displayed vertically. A moving image and a still image of an ultrasound image may be displayed horizontally or vertically.

In order to prevent hindrance against diagnosis with a moving image of an ultrasound image, an image may be displayed on the second display area 1920 smaller than a moving image of an ultrasound image displayed on the first display area 1910.

A moving image displayed on the second display area 1920 may include a moving image of at least one of a photoacoustic image and a superimposition image. A still image displayed on the second display area 1920 may only include a still image of at least one of a photoacoustic image and a superimposition image.

According to this display form, a moving image of a photoacoustic image can be displayed independently from a moving image of an ultrasound image, and a still image of a photoacoustic image can be displayed independently from a moving image of the ultrasound image in response to a save instruction. This can suppress hindrance against diagnosis with a moving image of an ultrasound image and allows auxiliary diagnosis with a photoacoustic image.

According to this embodiment, the first display area and the second display area may be assigned to display areas of one display apparatus, and the display areas may be assigned to a plurality of display apparatuses that are different from each other.

Having described a change of the display form in response to a save instruction according to this embodiment, the display form may be changed in response to an instruction other than an instruction involving saving. For example, in response to a still image display instruction for displaying a still image, change of the display form may be executed in response to a save instruction as described above.

Next, GUI (Graphic User Interface) examples for displaying an image as illustrated in FIGS. 20A to 20C will be described. FIG. 20A illustrates an ultrasound image, FIG. 20B illustrates a photoacoustic image, and FIG. 20C illustrates a superimposition image of an ultrasound image and a photoacoustic image. Though FIGS. 20A to 20C illustrate images exhibiting a two-dimensional spatial distribution, images exhibiting a three-dimensional spatial distribution may be displayed. The illustrated ultrasound images are assumed to be B mode images, but ultrasound images to be displayed are not limited thereto. The illustrated photoacoustic images are assumed to be images (such as a blood vessel image) exhibiting a spatial distribution of an optical absorption coefficient, but photoacoustic images to be displayed may not be limited thereto.

FIG. 21 illustrates a concrete example of the GUI to be displayed in the display unit 160.

A display area 2110 is a display area displaying a moving image of an ultrasound image. It is fundamentally assumed that a user may perform diagnosis by checking a moving image of an ultrasound image displayed on the display area.

The display area 2120 is an area displaying a thumbnail image corresponding to a time point of a still image display instruction given by using a FREEZE icon 2140. In the illustrated display example, a still image 2121 of an ultrasound image, a still image 2122 of a photoacoustic image, and a still image 2123 of a superimposition image of the ultrasound image and the photoacoustic image corresponding to the time point of a still image display instruction are displayed on the display area 2120. In this display example, three still images are displayed as thumbnails on the display area 2120 in response to one still image display instruction. When a user uses the input unit 170 to click the FREEZE icon 2140, a still image display instruction is given. Such a still image display instruction may not be given by using an icon on a GUI but may be given by using hardware such as a mechanical switch. Alternatively, for example, a touch screen may be used in the display unit 160. When a user touches the display area 2110 to give the still image display instruction, the still image display instruction may also function as a save instruction. In other words, an ultrasound image and a photoacoustic image corresponding to the still image display instruction may be saved in association.

In a case where it is difficult to display all thumbnail images on the display area 2020, such as a case where a plurality of still image display instructions is given, a user may change the thumbnail images to be displayed by operating a FORWARD IMAGE icon 2124. For comparison among still images of an ultrasound image, a photoacoustic image, and a superimposition image, the thumbnail image group to be displayed may be changed in response to every still image display instruction. For example, if an image forward instruction is given when a thumbnail image group corresponding to the first still image display instruction is being displayed, a thumbnail image group corresponding to the second still image display instruction may be displayed on the display area 2120. However, the rule for image forwarding is not limited thereto, but the image forwarding may be performed under any rule. For example, still images of an ultrasound image and a photoacoustic image corresponding to the first still image display instruction may be displayed, but the still image of the superimposition image may only be changed to a still image corresponding to the second still image display instruction. A user's operation instruction given through the FORWARD IMAGE icon may correspond to a switching instruction.

A display area 2130 is a display area displaying an image usable for setting information regarding an inspection objective and display parameters therefor. A region display area 2131 displays a region to be captured. In the illustrated display example, it is displayed that the region to be captured is the abdomen. The region to be captured, which is displayed on the region display area 2131, can be set based on examination order information.

A type display area 2132 is configured to display an image type of ultrasound image displayed on the display area 2110 or 2120. A user may use the input unit 170 to select an image type of ultrasound image to be displayed from a plurality of image types displayed on the type display area 2132. According to the illustrated display example, it is configured such that a user can select an image type from a B mode image, a doppler image, and an elastography image. The display example assumes a case where a B mode image is selected and displays such that the selection of a B mode image can be identified. Based on the image type selected on the type display area 2132, the control parameters (such as a pulse repetition frequency (PRF) and a pulse width of transmission ultrasonic waves) for transmission and reception of ultrasonic waves may be changed.

A type display area 2133 is configured to display an image type of photoacoustic image displayed on the display area 2120. A user may use the input unit 170 to select an image type of photoacoustic image to be displayed from a plurality of image types displayed on the type display area 2133. In the illustrated display example, it is configured such that a user can select an image type from an initial sound pressure image, an optical absorption coefficient image, and an oxygen saturation image. The display example assumes a case where an optical absorption coefficient image is selected and displays such that the selection of an optical absorption coefficient image can be identified. Based on the image type selected on the type display area 2133, control parameters (such as a wavelength, a pulse width, and an intensity of light) for light irradiation may be changed.

An ultrasound image and a photoacoustic image may be displayed in colors different from each other on the display unit 160. For example, in a case where an ultrasound image and a photoacoustic image are displayed in an overlapped manner, the photoacoustic image may be set to be displayed in a complementary color of the color of the ultrasound image for easy distinction between the ultrasound image and the photoacoustic image. For example, in a case where an ultrasound image and a photoacoustic image have image values at an identical pixel, the ultrasound image and photoacoustic image in their overlapped part may be displayed in different colors. A user may use the input unit 170 to click a coloring change area 2134 that is an icon for changing coloring of an ultrasound image or a photoacoustic image so that the corresponding coloring can be changed. The coloring of an image may be changed in response to a user's instruction given in a manner other than clicking on the coloring change area 2134 displayed on the display unit 160.

It may be configured such that the transmittance of an ultrasound image and a photoacoustic image in a superimposition image can be changed. For example, a user may use the input unit 170 to operate a slide bar 2135 horizontally to change the transmittance of an ultrasound image or a photoacoustic image. In the illustrated display example, it is configured such that the transmittance can be changed based on the position of the slide bar 2135.

A superimposition image having at least one image of an ultrasound image and a photoacoustic image having undergone an emphasizing process through a signal filter or an image filter may be displayed over the other one. For example, an edge emphasizing process may be performed on an ultrasound image, and the ultrasound image having an emphasized contour may be superimposed on a photoacoustic image for display. Alternatively, a blood vessel emphasizing process may be performed on a photoacoustic image, and the photoacoustic image showing an emphasized blood vessel may be superimposed on an ultrasound image for display.

After capturing processing performed by transmitting and receiving ultrasonic waves completes, a still image of the last frame of an ultrasound image or another image may be displayed on the display area 2110 without receiving a user's instruction. For example, based on a region to be captured, which is displayed on the region display area 2131, an image to be displayed on the display area 2110 may be set after completion of the capturing processing.

An image may be selected from thumbnail images displayed on the image display area 2120 by using the input unit 170 so that an enlarged image of the selected thumbnail may be displayed on the display area 2110. For example, one of still images 2121 to 2123 may be touched through a touch screen in the display unit 160 to select an image to be enlarged. One of the still images 2121 to 2123 may be swiped or flicked on the display area 2110 to select an image to be enlarged.

The illustrated display example displays solid lines indicating boundaries of the display areas for convenience of illustration, such boundaries may not be displayed.

FIG. 22 illustrates another concrete example of the GUI to be displayed on the display unit 160. Because a display area 2210, a display area 2230, and a FREEZE icon 2240 function in the same manner as those of the display area 2110, display area 2130, and FREEZE icon 2140 in FIG. 21, repetitive detail descriptions will be omitted. Repetitive detail descriptions on other same functions as those in FIG. 21 will also be omitted.

The display area 2220 is the same as the display area 2120 in that it displays a thumbnail image corresponding to a time point of a still image display instruction but the thumbnail image to be displayed is different from that in FIG. 21. The illustrated display example displays in the display area 2220 a still image 2221 of a superimposition image of an ultrasound image and a photoacoustic image exhibiting a spatial distribution of optical absorption coefficient and a still image 2222 of a superimposition image of the ultrasound image and a photoacoustic image exhibiting a spatial distribution of oxygen saturation. Both of the still image 2221 and the still image 2222 are thumbnail images corresponding to a time point of a display instruction for a common still image. In other words, this GUI horizontally displays superimposition images of different image types of photoacoustic image in response to a display instruction for one still image. Thus, a user can perform general diagnosis by using superimposition images of a plurality of photoacoustic images having diagnostic indices different from each other and an ultrasound image. Superimposition images of a plurality of photoacoustic images having diagnostic indices different from each other and an ultrasound image may be horizontally displayed as thumbnail images on the display area 2220. In this case, for example, a superimposition image of a B mode image and a photoacoustic image and a superimposition image of a doppler image and the photoacoustic image may be horizontally displayed as thumbnail images on the display area 2220. For example, a user may operate a FORWARD IMAGE icon 2223 to forward a thumbnail image group in response to every still image display instruction.

In response to a display instruction given when a moving image of a photoacoustic image is being displayed, a still image of an ultrasound image may be displayed, but the display of the moving image of the photoacoustic image may be continued on the other hand. The aforementioned first to fourth embodiments are based on a diagnosis with an ultrasound image and assume to provide a photoacoustic image as additional information. On the other hand, this form is based on a diagnosis with a photoacoustic image and is assumed to use an ultrasound image as additional information. According to this form, a save instruction can be received when a moving image of a photoacoustic image is being displayed, like a save instruction given when a moving image of an ultrasound image is being displayed according to the first to fourth embodiments. In other words, according to this form, an ultrasound image and a photoacoustic image (information derived from a photoacoustic signal) according to the first to fourth embodiments are interchanged. According to this form, hindrance against basic diagnosis with a photoacoustic image can be prevented, and at the same time a still image of an ultrasound image can be displayed.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-136104, filed Jul. 8, 2016, which is hereby incorporated by reference herein in its entirety.

1. An apparatus comprising:
a first obtaining unit configured to obtain an ultrasound image generated by transmitting and receiving ultrasonic waves to and from an object;
a second obtaining unit configured to obtain a photoacoustic image generated based on photoacoustic waves generated from light irradiated to the object; and
a display control unit configured to control to display the ultrasound image on a first display area of a display unit,
wherein the display control unit controls to
based on information representing a display instruction given when a moving image of the ultrasound image is being displayed, display a still image of a superimposition image of the ultrasound image and the photoacoustic image corresponding to a time point of the display instruction on a second display area different from the first display area in the display unit and, on the other hand, continue the display of the moving image of the ultrasound image on the first display area.

2. An apparatus comprising:
a first obtaining unit configured to obtain an ultrasound image generated by transmitting and receiving ultrasonic waves to and from an object;
a second obtaining unit configured to obtain a photoacoustic image generated based on photoacoustic waves generated from light irradiated to the object;
a display control unit configured to control to display a moving image of the ultrasound image on a first display area in a display unit and displays a moving image of a superimposition image of the ultrasound image and the photoacoustic image on a second display area different from the first display area in the display unit,
wherein the display control unit controls to
based on information representing a display instruction given when a moving image of the ultrasound image is being displayed, display a still image of a superimposition image of the ultrasound image and the photoacoustic image corresponding to a time point of the display instruction on the second display area and, on the other hand, continue the display of the moving image of the ultrasound image on the first display area.

3. An apparatus comprising:
a first obtaining unit configured to obtain an ultrasound image generated by transmitting and receiving ultrasonic waves to and from an object;
a second obtaining unit configured to obtain a photoacoustic image generated based on photoacoustic waves generated from light irradiated to the object;
a display control unit configured to control to display a moving image of the ultrasound image on a first display area in a display unit;
wherein the display control unit controls to
based on information representing a display instruction given when the moving image of the ultrasound image is being displayed, display a still image of a superimposition image of the ultrasound image and the photoacoustic image corresponding to a time point of the display instruction on the first display area for a predetermined period of time; and
after a lapse of the predetermined period of time from the display instruction, display a still image of the superimposition image on a second display area different from the first display area in the display unit and on the other hand, restarts the display of the moving image on the ultrasound image on the first display area.

4. The apparatus according to claim 1, wherein the display control unit controls to display a still image of a superimposition image of the ultrasound image and the photoacoustic image corresponding to a time point of the display instruction in a smaller size than that of the moving image of the ultrasound image.

5. The apparatus according to claim 1, wherein, based on information representing a plurality of display instructions given when a moving image of the ultrasound image is being displayed, the display control unit controls to horizontally display, on the second display area, still images of superimposition images of the ultrasound images and the photoacoustic images corresponding to time points of the plurality of display instructions.

6. The apparatus according to claim 1, wherein the display control unit controls to
based on information representing a plurality of display instructions given when a moving image of the ultrasound image is being displayed, generate still images of superimposition images of the ultrasound image and the photoacoustic image corresponding to time points of the plurality of display instructions; and
based on information representing a switching instruction, change the one to be displayed on the second display area of the still images of superimposition images of the ultrasound image and the photoacoustic image corresponding to the time points of the plurality of display instructions.

7. The apparatus according to claim 1, further comprising: a save control unit configured to save the ultrasound image and the photoacoustic image corresponding to the display instruction in association in a save unit based on information representing the display instruction.

8-17. (canceled)

18. The apparatus according to claim 2, wherein the display control unit controls to display a still image of a superimposition image of the ultrasound image and the photoacoustic image corresponding to a time point of the display instruction in a smaller size than that of the moving image of the ultrasound image.

19. The apparatus according to claim 2, wherein, based on information representing a plurality of display instructions given when a moving image of the ultrasound image is being displayed, the display control unit controls to horizontally display, on the second display area, still images of superimposition images of the ultrasound images and the photoacoustic images corresponding to time points of the plurality of display instructions.

20. The apparatus according to claim 2, wherein the display control unit controls to
based on information representing a plurality of display instructions given when a moving image of the ultrasound image is being displayed, generate still images of superimposition images of the ultrasound image and the photoacoustic image corresponding to time points of the plurality of display instructions; and
based on information representing a switching instruction, change the one to be displayed on the second display area of the still images of superimposition images of the ultrasound image and the photoacoustic image corresponding to the time points of the plurality of display instructions.

21. The apparatus according to claim 2, further comprising: a save control unit configured to save the ultrasound image and the photoacoustic image corresponding to the display instruction in association in a save unit based on information representing the display instruction.

22. The apparatus according to claim 2, wherein, at a time point of the display instruction, the display control unit controls to, based on information representing the display instruction, display a superimposition image of the ultrasound image displayed on the first display area and the photoacoustic image obtained at a time point neighboring in time to the ultrasound image on the second display area.

23. The apparatus according to claim 3, wherein the display control unit controls to display a still image of a superimposition image of the ultrasound image and the photoacoustic image corresponding to a time point of the display instruction in a smaller size than that of the moving image of the ultrasound image.

24. The apparatus according to claim 3, wherein, based on information representing a plurality of display instructions given when a moving image of the ultrasound image is being displayed, the display control unit controls to horizontally display, on the second display area, still images of superimposition images of the ultrasound images and the photoacoustic images corresponding to time points of the plurality of display instructions.

25. The apparatus according to claim 3, wherein the display control unit controls to
based on information representing a plurality of display instructions given when a moving image of the ultrasound image is being displayed, generate still images of superimposition images of the ultrasound image and the photoacoustic image corresponding to time points of the plurality of display instructions; and
based on information representing a switching instruction, change the one to be displayed on the second display area of the still images of superimposition images of the ultrasound image and the photoacoustic image corresponding to the time points of the plurality of display instructions.

26. The apparatus according to claim 3, further comprising: a save control unit configured to save the ultrasound image and the photoacoustic image corresponding to the display instruction in association in a save unit based on information representing the display instruction.

27. The apparatus according to claim 3, wherein, at a time point of the display instruction, the display control unit controls to, based on information representing the display instruction, display a superimposition image of the ultrasound image displayed on the first display area and the photoacoustic image obtained at a time point neighboring in time to the ultrasound image on the second display area.
